(19) United States
(12) Patent Application Publication
(10) Pub. No.: US 2026/0355324 A1
(43) Pub. Date: Nov. 12, 2026

(54) COVALENT COMPOUNDS AND USES THEREOF

(71) Applicants: The General Hospital Corporation, Boston, MA (US); President and Fellows of Harvard College, Cambridge, MA (US)

(72) Inventors: Anthony John Iafrate, Newton, MA (US); Liron Bar-Peled, Somerville, MA (US); Diane Yang, Malden, MA (US); Brian Liau; Stefan Harry, Cambridge, MA (US)

(21) Appl. No.: 19/120,182

(22) PCT Filed: Oct. 11, 2023
(86) PCT No.: PCT/US2023/076610

§ 371 Date: Apr. 10, 2025

Related U.S. Application Data
(60) Provisional application No. 63/415,279, filed on Oct. 11, 2022.
(60) Provisional application No. 63/506,966, filed on Jun. 8, 2023.

(51) Int. Cl. A61K 47/55; A61P 35/00; C40B 40/04

(52) U.S. Cl. CPC A61K 47/55; A61P 35/00; C40B 40/04

(57) ABSTRACT

The present document relates to covalent compounds and uses thereof, including methods of targeting or engaging one or more targets with a covalent compound. Also provided herein are methods of treating a disease using such a compound and methods of identifying one or more targets using such a compound.

### CROSS-REFERENCE TO RELATED APPLICATIONS

This application claims the benefit of U.S. Provisional Patent Application No. 63/415,279, filed on Oct. 11, 2022, and U.S. Provisional Patent Application No. 63/506,966, filed on Jun. 8, 2023, each of which is incorporated by reference herein in its entirety.

### FIELD

The present document relates to covalent compounds and uses thereof, including methods of targeting or engaging one or more targets with a covalent compound. Also provided herein are methods of treating a disease using such a compound and methods of identifying one or more targets using such a compound.

### BACKGROUND

Targeted therapies can be an effective modality in disease treatment. New approaches are needed to develop such therapies.

### SUMMARY

The present document relates to compounds (e.g., chemical compounds) that can be used to target or engage one or more targets. In particular embodiments, the compound includes at least one specificity group and at least one reactive moiety. The specificity group can be configured to engage with a specific target site of the target. The reactive moiety can be configured to bind one or more reactive sites of the target. One or more specificity groups, as well as one or more reactive moieties, may be present in the compound. The reactive moiety(ies) provided in the compound can be employed to form a covalent bond between the chemical compound and the target.

In one aspect, the present document features a method of targeting or engaging one or more targets with a chemical compound, the method including: binding a specificity group of the chemical compound to a specific target site, and reacting at least two reactive moieties of the chemical compound with respective reactive sites to form one or more covalent bonds.

In some embodiments, the one or more targets include the specific target site and at least two reactive sites. In some embodiments, the chemical compound includes a scaffold (e.g., any described herein) disposed between the specificity group (e.g., any described herein) and the at least two reactive moieties (e.g., any described herein).

In one aspect, the present document features a method of targeting or engaging one or more targets with a chemical compound, the method including: providing the chemical compound to the one or more targets, and incubating the chemical compound with the one or more targets under conditions configured to bind the specificity group to the specific target site and to react the at least two reactive moieties with the respective reactive site to form one or more covalent bonds.

In some embodiments, the chemical compound includes: a specificity group configured to interact with a specific target site of the one or more targets, and at least two reactive moieties. In some embodiments, each of the at least two reactive moieties is configured to react with a respective reactive site of the one or more targets.

In one aspect, the present document features a method of targeting or engaging one or more targets with a chemical compound, the method including: providing an effective amount of the chemical compound.

In some embodiments, the chemical compound includes a structure having Formula (I):

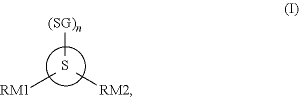

wherein:
   S includes a scaffold;
   each SG includes, independently, a specificity group configured to interact with a respective specific target site of the one or more targets;
   each of RM1 and RM2 includes, independently, a reactive moiety configured to react with a respective reactive site of the one or more targets to form one or more covalent bonds; and
   n is an integer of 1 or more.

In one aspect, the present document features a method of identifying one or more targets, the method including: providing a chemical compound to a test sample; and identifying a biological target bound to the chemical compound. In some embodiments, the chemical compound includes a structure having Formula (I) (e.g., as described herein).

In one aspect, the present document features a method of treating a disease, the method including: administering a therapeutically effective amount of a chemical compound to a subject in need thereof. In some embodiments, the chemical compound includes a structure having Formula (I) (e.g., as described herein).

In some embodiments, the disease includes cancer (e.g., non-small cell lung cancer (NSCLC), lung adenocarcinoma, lung cancer, breast cancer, or colorectal cancer). In some embodiments, the cancer is characterized by a presence of an EML4-ALK fusion gene, an EML4-ALK fusion protein, an EML4 fusion gene, an EML4 fusion protein, an ALK fusion gene, an ALK fusion protein, or a variant thereof.

In one aspect, the present document features a chemical compound including a structure having Formula (II):

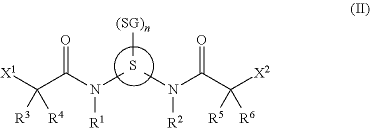

or a pharmaceutically acceptable salt thereof;
wherein:
   S includes a scaffold;
   each SG includes, independently, a specificity group configured to interact with a respective specific target site of one or more targets;
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n is an integer of 1 or more, and
   wherein the compound is not 2-chloro-N-[[(2-chloroacetyl)amino]-(3-methoxy-4-phenylmethoxyphenyl)methyl]acetamide.

In one aspect, the present document features a chemical compound including a structure having Formula (II):

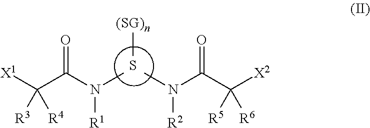

or a pharmaceutically acceptable salt thereof;
wherein:
   S includes a scaffold;
   each SG includes, independently, a specificity group configured to interact with a respective specific target site of one or more targets;
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n is an integer of 1 or more, and
   wherein at least one of S, SG, R¹, R², R³, R⁴, R⁵, or R⁶ includes an E3 ubiquitin ligase (E3) ligand.

In one aspect, the present document features a library including a plurality of test compounds, wherein at least one of the plurality of test compounds includes a structure having Formula (II):

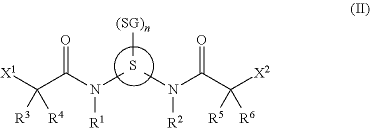

or a pharmaceutically acceptable salt thereof;
wherein:
   S includes a scaffold;
   each SG includes, independently, a specificity group configured to interact with a respective specific target site of one or more targets;
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n is an integer of 1 or more, and
   wherein the compound is not 2-chloro-N-[[(2-chloroacetyl)amino]-(3-methoxy-4-phenylmethoxyphenyl)methyl]acetamide; and
   optionally wherein at least one of S, SG, R¹, R², R³, R⁴, R⁵, or R⁶ includes an E3 ubiquitin ligase (E3) ligand.

In any embodiment herein, the one or more targets includes a protein, a peptide, a fusion protein, or a constitutively active receptor protein (e.g., any described herein).

In any embodiment herein, the one or more targets is, independently, selected from the group consisting of EML4-ALK, EML4, EML4 fusion protein, ALK fusion protein, PSME1, PSME2, TMX1, TXN, TJP2, PDIA6, P4HB, AGR2, PDIA3, PDIA4, PPFIAI, SMARCC2, GCLC, NPEPPS, PDCD61P, PRDX1, ERO1A, PCBP1, CTTN, PRDX2, PRDX3, RARSI, TXNDC17, RPL21, S100A2, KIF5B, and RPL18.

In any embodiment herein, the one or more targets includes an ALK fusion protein having one or more mutations. In some embodiments, the one or more mutations includes 1151T-ins, L1152R/P, C1156Y, 11171T/N/S, F1174L/C/V, V1180L, L1196M/F, L1198P, G1202R/del, D1203N, S1206Y, E1210K, or G1269A, or a combinations of any of these.

In any embodiment herein, the one or more targets includes a coiled-coil domain.

In any embodiment herein, the specific target site includes the coiled-coil domain.

In any embodiment herein, the one or more targets includes one or more cysteines.

In any embodiment herein, the at least two reactive sites includes at least two cysteines.

In any embodiment herein, the reactive site includes the cysteine.

In any embodiment herein, the one or more targets is present within a cell, a culture, a tissue sample, a lysate, or a test sample.

In any embodiment herein, the method, the chemical compound, and/or the test compound thereby inhibits the one or more targets.

In any embodiment herein, the method, the chemical compound, and/or the test compound thereby inhibits signaling of the one or more targets. In some embodiments, the signaling includes signaling of kinase activity.

In any embodiment herein, the chemical compound includes a compound of having any one of Formulas (I), (Ia), (II), (IIa), (III), (IV), (V), and (1)-(25)).

In any embodiment herein, the test compound includes a compound of having any one of Formulas (I), (Ia), (II), (IIa), (III), (IV), (V), and (1)-(25)).

A compound can include a specificity group and/or a scaffold. In any embodiment herein, the specificity group and/or the scaffold, if present, includes an E3 ubiquitin ligase (E3) ligand. In some embodiments, a linker is disposed between the specificity group and the E3 ligand and/or between the scaffold and the E3 ligand (e.g., any described herein).

In any embodiments, the specificity group, the scaffold, SG, SG′, and/or S, if present, is independently substituted with one or more of the following groups: halo, hydroxyl, nitro, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

A compound can include a specificity group (e.g., SG and/or SG′). In any embodiment herein, the specificity group, SG, and/or SG′, if present, includes an aromatic group. In some embodiments, the aromatic group includes an optionally substituted aryl, optionally substituted phenyl, optionally substituted benzyl, optionally substituted biphenyl, optionally substituted naphthyl, optionally substituted pyrenyl, optionally substituted indenyl, optionally substituted fluorenyl, optionally substituted phenalenyl, optionally substituted phenanthryl, optionally substituted anthryl, optionally substituted triphenylenyl, optionally substituted tetracenyl, optionally substituted acenaphthenyl, optionally substituted carbazolyl (dibenzopyrrolyl), optionally substituted dibenzofuranyl, optionally substituted quinolinyl, optionally substituted isoquinolinyl, optionally substituted indolyl, optionally substituted benzimidazolyl, or optionally substituted thiazolyl.

In any embodiments, the specificity group, SG, and/or SG′, if present, independently includes optionally substituted phenyl, optionally substituted benzyl, or optionally substituted pyrenyl.

A compound can include a scaffold (e.g., S). In any embodiment herein, the scaffold or S, if present, includes a monovalent, bivalent, trivalent, tetravalent, or pentavalent linker. In some embodiments, the linker includes a covalent bond, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group.

A compound can include a reactivity moiety (e.g., RM, RM1, and/or RM2). In any embodiment herein, the reactive moiety, the at least two reactive moieties, RM1, and/or RM2, if present, independently includes an electrophilic group or a leaving group.

A compound can include an electrophilic group and/or a leaving group. In any embodiment herein, the electrophilic group or the leaving group includes optionally substituted haloacetyl, optionally substituted haloacetylamido, optionally substituted acryloyl, optionally substituted acrylamido, optionally substituted alkenyl, optionally substituted alkynyl, optionally substituted oxiranyl, halo, cyano, isocyanato, isothiocyanato, optionally substituted alkenylsulfonyl, halosulfonyl, optionally substituted maleimidyl, optionally substituted alkoxy, optionally substituted thioalkoxy, optionally substituted thioaryloxy, or thiol.

A compound can include an aromatic group. In any embodiment herein, each of A1, A2, Ar₁, and Ar₂, includes, independently, one or more aromatic groups (e.g., any described herein).

A compound can include a ligand. In any embodiment herein, the ligand includes a binding ligand or a targeting ligand. In some embodiments, the ligand includes an E3 ubiquitin ligase (E3) ligand (e.g., any described herein). In some embodiments, the E3 ligand is a ligand for Skp1-Cullin-F box complex (SCFB-TrCP), von Hippel-Lindau (VHL), murine double minute 2 (MDM2), an inhibitor of apoptosis proteins (IAP), or cereblon (CRBN). In some embodiments, the E3 ligand includes a thalidomide-based ligand, a pomalidomide-based ligand, a 4-hydroxythalidomide-based ligand, a lenalidomide-based ligand, a VH032-based ligand, a VHL ligand 1 (VHL A1)-based ligand, a nutlin-3-based ligand, an idasanutlin-based ligand, a bestatin-based ligand, a methyl bestatin-based ligand, or a LCL-161-based ligand.

A compound can include a linker. In any embodiment herein, the linker includes a covalent bond, oxy, carbonyl, imino, nitrilo, an atom, an optionally substituted alkylene group, an optionally substituted alkyleneoxy group, an optionally substituted heteroalkylene group, an optionally substituted arylene group, or an optionally substituted heteroarylene group, which can further optionally include a click chemistry signature. In some embodiments, each of L, L₁, L₁ₐ, L_{1b}, L_{1c}, L₂, L₂ₐ, and L_{2b} includes, independently, a linker (e.g., any described herein). In some embodiments, a linker is disposed between at least one of the S, SG, R¹, R², R³, R⁴, R⁵, or R⁶ and a ligand (e.g., an E3 ligand).

In any embodiment herein, the linker includes -L^{A}-L^{B}-L^{C}-, and each of L^{A}, L^{B}, and L^{C} includes, independently, a linker. In some embodiments, each of L^{A} and L^{B} is, independently, a covalent bond, oxy, carbonyl, carbonyloxy, imino, nitrilo, sulfo, an atom, an optionally substituted alkylene group, an optionally substituted alkyleneoxy group, an optionally substituted heteroalkylene group, an optionally substituted arylene group, or an optionally substituted heteroarylene group, which can further optionally include a click chemistry signature. In some embodiments, L^{C} includes a click chemistry signature.

In any embodiment herein, each of R¹, R², R³, R⁴, R^{4a}, R^{4b}, R⁵, R⁶, R^{6a}, R^{6b}, and R^{L} is, independently, H, halo, optionally substituted C₁₋₁₂ aliphatic, or optionally substituted C₁₋₁₂ heteroaliphatic.

In any embodiment herein, each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group.

In any embodiment herein, each of R⁷ is, independently, H, optionally substituted aliphatic, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano.

In any embodiment herein, each of R⁸ is, independently, H, optionally substituted aliphatic, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano.

In any embodiment herein, each of R⁷ is, independently, halo, hydroxyl, nitro, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, each of R⁸ is, independently, halo, hydroxyl, nitro, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, the chemical compound and/or the test compound is selected from SH001, SH002, SH003, SH004, SH005, SH006, SH007, SH008, SH009, SH010, SH011, SH012, SH013, SH014, SH015, SH016, SH017, SH018, SH019, SH020, SH021, SH022, SH023, SH024, SH025, SH026, SH027, SH028, SH029, SH030, SH031, SH032, SH033, SH034, SH035, SH036, SH037, SH038, SH039, SH040, SH041, SH042, SH043, SH044, SH045, SH046, SH047, SH048, SH049, SH050, SH051, SH052, SH053, or a pharmaceutically acceptable salt thereof.

In any embodiment herein, the chemical compound and/or the test compound is selected from SH002, SH003, SH004, SH005, SH006, SH009, SH012, SH013, SH014, SH015, SH016, SH017, SH018, SH019, SH020, SH021, SH022, SH023, SH028, SH031, SH032, SH034, SH036, SH037, SH038, SH039, SH040, SH043, or a pharmaceutically acceptable salt thereof.

In any embodiment herein, the chemical compound and/or the test compound is selected from SH002, SH003, SH004, SH005, SH013, SH014, SH015, SH017, SH018, SH019, SH020, SH021, SH022, SH023, SH028, SH031, SH032, SH034, SH036, SH037, or a pharmaceutically acceptable salt thereof.

In any embodiment herein, the chemical compound and/or the test compound is selected from SH013, SH031, SH038, SH039, SH043, or a pharmaceutically acceptable salt thereof. Additional details are provided herein.

### DEFINITIONS

As used herein, the term “about” means +/−10% of any recited value. As used herein, this term modifies any recited value, range of values, or endpoints of one or more ranges.

By “attach,” “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, x bond interactions, hydrophobic interactions, inclusion complexes, clathration, van der Waals interactions, and combinations thereof.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof (e.g., cycloaliphatic), and further including straight- and branched-chain arrangements, and all stereo and position isomers as well.

By “alkanoyl” or “acyl,” as used interchangeably herein, is meant-C(O)—R, in which R is hydrogen, an optionally substituted aliphatic group, or an optionally substituted alkyl group, as defined herein. The aliphatic or alkyl groups can be substituted or unsubstituted. For example, the aliphatic or alkyl groups can be substituted with one or more substitution groups, as described herein for alkyl.

By “alkanoyloxy” is meant —O—C(O)—R, in which R is hydrogen, an optionally substituted aliphatic group, or an optionally substituted alkyl group, as defined herein. The aliphatic or alkyl groups can be substituted or unsubstituted. For example, the aliphatic or alkyl groups can be substituted with one or more substitution groups, as described herein for alkyl.

By “alkaryl” is meant —Ar-Ak, where Ar is an optionally substituted arylene group and Ak is an optionally substituted aliphatic group or optionally substituted alkyl group, as described herein. The arylene, aliphatic, and alkyl groups can be substituted or unsubstituted. For example, the arylene, aliphatic, and alkyl groups can be substituted with one or more substitution groups, as described herein for alkyl or aryl. Exemplary unsubstituted alkaryl groups include C₁₋₃ alkyl-C₄₋₁₈ aryl, C₁₋₆ alkyl-C₄₋₁₈ aryl, and C₁₋₁₂ alkyl-C₄₋₁₈ aryl groups.

By “alkenyl” or “alkene” is meant an optionally substituted C₂₋₂₄ alkyl group having one or more double bonds, as well as a compound having such a group. In some embodiments, the unsaturated monovalent hydrocarbon can be derived from removing one hydrogen atom from one carbon atom of a parent alkene. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl.

By “alkenyloxy” is meant-OR, where R is an optionally substituted alkenyl group, as described herein.

By “alkenylsulfonyl” is meant —SO₂R, where R is an optionally substituted alkenyl group, as described herein.

By “alkoxy” is meant-OR, where R is an optionally substituted aliphatic group or an optionally substituted alkyl group, as described herein. Exemplary alkoxy groups include, but are not limited to, methoxy, ethoxy, n-propoxy, isopropoxy, n-butoxy, t-butoxy, sec-butoxy, n-pentoxy, haloalkoxy (e.g., trihaloalkoxy, such as trifluoromethoxy, dihaloalkoxy, such as difluoromethoxy, monohaloalkoxy, and the like), etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkoxycarbonyl” is meant —C(O)—OR, where R is an optionally substituted aliphatic group or an optionally substituted alkyl group, as described herein. The alkoxycarbonyl group can be substituted or unsubstituted. For example, the alkoxycarbonyl group can be substituted with one or more substitution groups, as described herein for alkyl. Exemplary unsubstituted alkoxycarbonyl groups include C₁₋₃, C₁₋₆, C₁₋₁₂, C₁₋₁₆, C₁₋₁₈, C₁₋₂₀, or C₁₋₂₄ alkoxycarbonyl groups.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₂₋₈ alkenyl; (2) C₂₋₈ alkynyl; (3) C₁₋₆ alkoxy (e.g., —OR^{Y}); (4) C₁₋₆ alkylsulfinyl (e.g., —S(O)R^{Y}); (5) C₁₋₆ alkylsulfonyl (e.g., —SO₂R^{Y}); (6) amino; (7) aryl (e.g., C₄₋₁₈ aryl); (8) arylalkoxy (e.g., —OR^{Z}Ar^{Z}); (9) aryloyl (e.g., —C(O)Ar₂); (10) azido (—N₃); (11) carboxyaldehyde (—C(O)H); (12) carboxyl (—C(O)OH); (13) C₃₋₈ cycloalkyl; (14) halo; (15) heterocyclyl (e.g., C₁₋₁₈ heterocyclyl including one or more heteroatoms, such as N, O, S, and P); (16) heterocyclyloxy (e.g., —OHet^{Z}); (17) heterocyclyloyl (e.g., —C(O)Het^{Z}); (18) hydroxyl; (19)N-protected amino; (20) nitro (—NO₂); (21) oxo (═O); (22) C₃₋₈ spirocyclyl; (23) C₁₋₆ thioalkoxy (e.g., —SR); (24) thiol (—SH); (25) —CO₂R^{A} (e.g., —C(O)—OR^{A} or —O—C(O)—R^{A}), where R^{A} is selected from the group consisting of (a) hydrogen, (b) C₁₋₆ alkyl, (c) C₄₋₁₈ aryl, and (d) C₁₋₆ alk-C₄₋₁₈ aryl; (26) —C(O)NR^{B}R^{C}, where each of R^{B} and R^{C} is, independently, selected from the group consisting of (a) hydrogen, (b) C₁₋₆ alkyl, (c) C₄₋₁₈ aryl, and (d) C₁₋₆ alk-C₄₋₁₈ aryl; (27) —SO₂R^{D}, where R^{D} is selected from the group consisting of (a) C₁₋₆ alkyl, (b) C₄₋₁₈ aryl, and (c) C₁₋₆ alk-C₄₋₁₈ aryl; (28) —SO₂NRERF, where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; and (29) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃₋₈ cycloalkyl, and (i) C₁₋₆ alk-C₃₋₈ cycloalkyl, where in one embodiment, no two groups are bound to the nitrogen atom through a carbonyl group or a sulfonyl group (e.g., where R^{Y} is alkyl (e.g., C₁₋₆ alkyl), R^{Z} is alkylene (e.g., C₁₋₆ alkylene), Ar^{Z} is aryl (e.g., C₄₋₁₈ aryl), and Het^{Z} is heterocyclyl (e.g., C₁₋₁₈ heterocyclyl including one or more heteroatoms, such as N, O, S, and P), as defined herein). The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent, trivalent, tetravalent, etc.) form of an aliphatic group or an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkyleneoxy” is meant —OL— or —LO—, wherein L is an optionally substituted alkylene group, as described herein. Exemplary alkyleneoxy groups include methyleneoxy, ethyleneoxy, propyleneoxy, butyleneoxy, etc. In some embodiments, the alkyleneoxy group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkyleneoxy group. The alkyleneoxy group can be branched or unbranched. The alkyleneoxy group can also be substituted or unsubstituted. For example, the alkyleneoxy group can be substituted with one or more substitution groups, as described herein for alkyl.

By “alkylsulfonyl” is meant —SO₂R, where R is an optionally substituted aliphatic group or an optionally substituted alkyl group, as described herein. The alkylsulfonyl group can be substituted or unsubstituted. For example, the alkylsulfonyl group can be substituted with one or more substitution groups, as described herein for alkyl. Exemplary unsubstituted alkylsulfonyl groups include C₁₋₃, C₁₋₆, C₁₋₁₂, C₁₋₁₆, C₁₋₁₈, C₁₋₂₀, or C₁₋₂₄ alkylsulfonyl groups.

By “alkynyl” or “alkyne” is meant an unsaturated monovalent hydrocarbon having at least two carbon atom to 50 carbon atoms (C₂₋₅₀) and at least one carbon-carbon triple bond, as well as a compound having such a group. In some embodiments, the unsaturated monovalent hydrocarbon can be derived from removing one hydrogen atom from one carbon atom of a parent alkyne. An alkynyl group can be branched, straight-chain, or cyclic (e.g., cycloalkynyl); and can include two to 25 carbon atoms (C₂₋₂₅) or two to ten carbon atoms (C₂-10). An exemplary alkynyl includes an optionally substituted C₂₋₂₄ alkyl group having one or more triple bonds. The alkynyl group can be cyclic or acyclic and is exemplified by ethynyl, 1-propynyl, and the like. The alkynyl group can be monovalent or multivalent (e.g., bivalent) by removing one or more hydrogens to form appropriate attachment to the parent molecular group or appropriate attachment between the parent molecular group and another substitution. The alkynyl group can also be substituted or unsubstituted. For example, the alkynyl group can be substituted with one or more substitution groups, as described herein for alkyl.

By “alkynyloxy” is meant-OR, where R is an optionally substituted alkynyl group, as described herein.

By “amino” is meant —NR^{N1}R^{N2}, where each of R^{N1} and R^{N2} is, independently, H, optionally substituted aliphatic, alkyl, heteroaliphatic, heteroalkyl, aromatic, or aryl; or where R^{N1} and R^{N2}, taken together with the nitrogen atom to which each are attached, form a heterocyclyl group. The amino group can be substituted or unsubstituted. For example, the amino group can be substituted with one or more substitution groups, as described herein for alkyl or aryl.

By “aralkyl” is meant -Ak-Ar, where Ak is an optionally substituted alkylene group and Ar is an optionally substituted aromatic group or optionally substituted aryl group, as described herein. The alkylene, aromatic, and aryl groups can be substituted or unsubstituted. For example, the alkylene, aromatic, and aryl groups can be substituted with one or more substitution groups, as described herein for alkyl or aryl. Exemplary unsubstituted aralkyl groups include C₄₋₁₈ aryl-C₁₋₃ alkyl, C₄₋₁₈ aryl —C₁₋₆ alkyl, and C₄₋₁₈ aryl-C₁₋₁₂ alkyl groups.

By “aralkoxy” is meant —O-Ak-Ar, where Ak is an optionally substituted alkylene group and Ar is an optionally substituted aromatic group or optionally substituted aryl group, as described herein. The alkylene, aromatic, and aryl groups can be substituted or unsubstituted. For example, the alkylene, aromatic, and aryl groups can be substituted with one or more substitution groups, as described herein for alkyl or aryl. Exemplary unsubstituted aralkoxy groups include C₄₋₁₈ aryl-C₁₋₃ alkoxy, C₄₋₁₈ aryl —C₁₋₆ alkoxy, and C₄₋₁₈ aryl-C₁₋₁₂ alkoxy groups.

By “aralkanoyl” is meant —C(O)-Ak-Ar, where Ak is an optionally substituted alkylene group and Ar is an optionally substituted aromatic group or optionally substituted aryl group, as described herein. The alkylene, aromatic, and aryl groups can be substituted or unsubstituted. For example, the alkylene, aromatic, and aryl groups can be substituted with one or more substitution groups, as described herein for alkyl or aryl. Exemplary unsubstituted aralkanoyl groups include C₄₋₁₈ aryl-C₁₋₃ alkanoyl, C₄₋₁₈ aryl —C₁₋₆ alkanoyl, and C₄₋₁₈ aryl-C₁₋₁₂ alkanoyl groups.

By “aralkanoyloxy” is meant —O—C(O)-Ak-Ar, where Ak is an optionally substituted alkylene group and Ar is an optionally substituted aromatic group or optionally substituted aryl group, as described herein. The alkylene, aromatic, and aryl groups can be substituted or unsubstituted. For example, the alkylene, aromatic, and aryl groups can be substituted with one or more substitution groups, as described herein for alkyl or aryl. Exemplary unsubstituted aralkanoyloxy groups include C₄₋₁₈ aryl-C₁₋₃ alkanoyloxy, C₄₋₁₈ aryl-C₁₋₆ alkanoyl, and C₄₋₁₈ aryl-C₁₋₁₂ alkanoyloxy groups.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized re-electron system. Typically, the number of out of plane re-electrons corresponds to the Huckel rule (4n+2). The point of attachment to the parent structure typically is through an aromatic portion of the condensed ring system. The term “aromatic” also includes “heteroaromatic,” as described herein.

By “aryl” is meant a group that contains any carbon-based aromatic group including, but not limited to, benzyl, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O) Ak, in which Ak is an alkyl group, as defined herein); (2) C₁₋₆ alkyl; (3) C₁₋₆ alkoxy (e.g., —OAk, in which Ak is an alkyl group, as defined herein); (4) C₁₋₆ alkoxy-C₁₋₆ alkyl (e.g., an alkyl group, which is substituted with an alkoxy group -OAk, in which Ak is an alkyl group, as defined herein); (5) C₁₋₆ alkylsulfinyl (e.g., —S(O) Ak, in which Ak is an alkyl group, as defined herein); (6) C₁₋₆ alkylsulfinyl-C₁₋₆ alkyl (e.g., an alkyl group, which is substituted by an alkylsulfinyl group-S(O) Ak, in which Ak is an alkyl group, as defined herein); (7) C₁₋₆ alkylsulfonyl (e.g., —SO₂Ak, in which Ak is an alkyl group, as defined herein); (8) C₁₋₆ alkylsulfonyl-C₁₋₆ alkyl (e.g., an alkyl group, which is substituted by an alkylsulfonyl group -SO₂Ak, in which Ak is an alkyl group, as defined herein); (9) aryl; (10) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., meant an alkyl group, as defined herein, substituted by an amino group); (12) heteroaryl; (13) C₁₋₆ alk-C₄₋₁₈ aryl (e.g., —A^{L} Ar, in which A^{L} is an alkylene group and Ar is an aryl group, as defined herein); (14) aryloyl (e.g., —C(O)Ar, in which Ar is an aryl group, as defined herein); (15) azido (e.g., an —N₃ group); (16) cyano (e.g., a —CN group); (17) C₁₋₆ azidoalkyl (e.g., a —N₃ azido group attached to the parent molecular group through an alkyl group, as defined herein); (18) carboxyaldehyde (e.g., a —C(O)H group); (19) carboxyaldehyde-C₁₋₆ alkyl (e.g., —A^{L}C(O)H, in which A^{L} is an alkylene group, as defined herein); (20) C₃₋₈ cycloalkyl; (21) C₁₋₆ alk-C₃₋₈ cycloalkyl (e.g., —A^{L}Cy, in which A^{L} is an alkylene group and Cy is a cycloalkyl group, as defined herein); (22) halo (e.g., F, Cl, Br, or I); (23) C₁₋₆ haloalkyl (e.g., an alkyl group, as defined herein, substituted with one or more halo); (24) heterocyclyl; (25) heterocyclyloxy (e.g., —OHet, in which Het is a heterocyclyl group); (26) heterocyclyloyl (e.g., —C(O)Het, in which Het is a heterocyclyl group); (27) hydroxyl (e.g., a —OH group); (28) C₁₋₆ hydroxyalkyl (e.g., an alkyl group, as defined herein, substituted by one to three hydroxyl groups, with the proviso that no more than one hydroxyl group may be attached to a single carbon atom of the alkyl group); (29) nitro (e.g., an —NO₂ group); (30) C₁₋₆ nitroalkyl (e.g., an alkyl group, as defined herein, substituted by one to three nitro groups); (31)N-protected amino; (32)N-protected amino-C₁₋₆ alkyl; (33) oxo (e.g., an ═O group); (34) C₁₋₆ thioalkoxy (e.g., —SAK, in which Ak is an alkyl group, as defined herein); (35) thio-C₁₋₆ alkoxy-C₁₋₆ alkyl (e.g., an alkyl group, which is substituted by an thioalkoxy group-SAK, in which Ak is an alkyl group, as defined herein); (36) —(CH₂), CO₂R^{A} (e.g., —(CH₂)ᵣ—C(O)—OR^{A} or —(CH₂)ᵣO—C(O)—R^{A}), where r is an integer of from zero to four, and R^{A} is selected from the group consisting of (a) hydrogen, (b) C₁₋₆ alkyl, (c) C₄₋₁₈ aryl, and (d) C₁₋₆ alk-C₄₋₁₈ aryl; (37)—(CH₂)ᵣCONR^{B}R^{C}, where r is an integer of from zero to four and where each R^{B} and R^{C} is independently selected from the group consisting of (a) hydrogen, (b) C₁₋₆ alkyl, (c) C₄₋₁₈ aryl, and (d) C₁₋₆ alk-C₄₋₁₈ aryl; (38) (CH₂)ᵣSO₂R^{D}, where r is an integer of from zero to four and where R^{D} is selected from the group consisting of (a) C₁₋₆ alkyl, (b) C₄₋₁₈ aryl, and (c) C₁₋₆ alk-C₄₋₁₈ aryl; (39) (CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; (40)—(CH₂)ᵣNR^{G}R^{H}, where r is an integer of from zero to four and where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃₋₈ cycloalkyl, and (i) C₁₋₆ alk-C₃₋₈ cycloalkyl, wherein in one embodiment, no two groups are bound to the nitrogen atom through a carbonyl group or a sulfonyl group; (41) thiol; (42) perfluoroalkyl (e.g., an alkyl group, as defined herein, having each hydrogen atom substituted with a fluorine atom); (43) perfluoroalkoxy (e.g., —OR_{f}, in which R_{f} is an alkyl group, as defined herein, having each hydrogen atom substituted with a fluorine atom); (44) aryloxy (e.g., —OAr, where Ar is an optionally substituted aryl group, as described herein); (45) cycloalkoxy (e.g., —OCy, in which Cy is a cycloalkyl group, as defined herein); (46) cycloalkylalkoxy (e.g., —OA^{L}Cy, in which A^{L} is an alkylene group and Cy is a cycloalkyl group, as defined herein); and (47) arylalkoxy (e.g., —OA^{L}Ar, in which A^{L} is an alkylene group and Ar is an aryl group, as defined herein). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylene” is meant a multivalent (e.g., bivalent, trivalent, tetravalent, etc.) form of an aromatic group or aryl group, as described herein. Exemplary arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for alkyl or aryl.

By “aryloxy” is meant —O—Ar, where Ar is an optionally substituted aromatic group or optionally substituted aryl group, as described herein. The aromatic or aryl group can be substituted or unsubstituted. For example, the aromatic or aryl group can be substituted with one or more substitution groups, as described herein for alkyl or aryl. Exemplary unsubstituted aryloxy groups include C₄₋₁₈, C₄₋₁₄, C₄₋₁₂, C₄₋₁₀, C₄₋₁₈, C₆₋₁₄, C₆₋₁₂, or C₆₋₁₀ aryloxy groups.

By “aryloxycarbonyl” is meant —C(O)—O—Ar, where Ar is an optionally substituted aromatic group or optionally substituted aryl group, as described herein. The aromatic or aryl group can be substituted or unsubstituted. For example, the aromatic or aryl group can be substituted with one or more substitution groups, as described herein for alkyl or aryl. Exemplary unsubstituted aryloxycarbonyl group include C₅₋₁₈, C₅₋₁₄, C₅₋₁₂, C₅₋₁₀, C₇₋₁₈, C₇₋₁₄, C₇₋₁₂, or C₇₋₁₀ aryloxycarbonyl groups.

By “aryloyl” is meant —C(O)—Ar, where Ar is an optionally substituted aromatic group or optionally substituted aryl group, as described herein. The aromatic or aryl group can be substituted or unsubstituted. For example, the aromatic or aryl group can be substituted with one or more substitution groups, as described herein for alkyl or aryl. Exemplary unsubstituted aryloyl group include C₅₋₁₈, C₅₋₁₄, C₅₋₁₂, C₅₋₁₀, C₇₋₁₈, C₇₋₁₄, C₇₋₁₂, or C₇₋₁₀ aryloyl groups.

By “aryloyloxy” is meant —O—C(O)—Ar, where Ar is an optionally substituted aromatic group or optionally substituted aryl group, as described herein. The aromatic or aryl group can be substituted or unsubstituted. For example, the aromatic or aryl group can be substituted with one or more substitution groups, as described herein for alkyl or aryl. Exemplary unsubstituted aryloyloxy group include C₅₋₁₈, C₅₋₁₄, C₅₋₁₂, C₃₋₁₀, C₇₋₁₈, C₇₋₁₄, C₇₋₁₂, or C₇₋₁₀ aryloyloxy groups.

By “arylsulfonyl” is meant —SO₂R, where R is an optionally substituted aromatic group or an optionally substituted aryl group, as described herein. The arylsulfonyl group can be substituted or unsubstituted. For example, the arylsulfonyl group can be substituted with one or more substitution groups, as described herein for alkyl or aryl. Exemplary unsubstituted arylsulfonyl groups include C₄₋₁₈, C₄₋₁₄, C₄₋₁₂, C₄₋₁₀, C₄₋₁₈, C₆₋₁₄, C₆₋₁₂, or C₆₋₁₀ arylsulfonyl groups.

By “carbonyl” and the suffix “oyl” is meant —C(O)—.

By “carbonyloxy” is meant —O—C(O)— or —C(O)—O—.

As used herein, the term “click chemistry signature” refers to a plurality of atoms disposed between and covalently linking entity A and entity B, in which the click chemistry signature is formed as the product of a click reaction that links entity A and entity B. In certain instances, the click chemistry signature has the structure of a click chemistry signature that is formed as the product of a click reaction that links entity A and entity B, but is not limited to a click chemistry signature made by any particular process, e.g., not limited to a click chemistry signature formed by a click reaction, but can be formed or provided by another process. In one non-limiting instance, the click chemistry signature is an alkyne/azide click chemistry signature, e.g., the click chemistry signature comprises a triazole (e.g., a 1,2,3-triazole).

By “cyano” is meant-CN.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.] heptyl and the like. The cycloalkyl group can also be substituted or unsubstituted. For example, the cycloalkyl group can be substituted with one or more groups including those described herein for alkyl.

By “halo” is meant F, Cl, Br, or I.

By “haloalkyl” is meant an optionally substituted alkyl, as defined herein, having one or more halo substituents.

By “haloalkoxy” is meant —OR, where R is haloalkyl, as described herein. The haloalkoxy group can be substituted or unsubstituted. For example, the haloalkoxy group can be substituted with one or more substitution groups, as described herein for alkyl. Exemplary unsubstituted haloalkoxy groups include C₁₋₃, C₁₋₆, C₁₋₁₂, C₁₋₁₆, C₁₋₁₈, C₁₋₂₀, or C₁₋₂₄ haloalkoxy groups.

By “heteroaliphatic” is meant an aliphatic group, as defined herein, including at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

By “heteroalkyl,” “heteroalkenyl,” and “heteroalkynyl” is meant an alkyl, alkenyl, or alkynyl group (which can be branched, straight-chain, or cyclic), respectively, as defined herein, including at least one heteroatom to 20 heteroatoms, such as one to 15 heteroatoms, or one to 5 heteroatoms, which can be selected from, but not limited to, oxygen, nitrogen, sulfur, silicon, boron, selenium, phosphorous, and oxidized forms thereof within the group.

By “heteroalkylene” is meant an alkylene group, as defined herein, containing one, two, three, or four non-carbon heteroatoms (e.g., independently selected from the group consisting of nitrogen, oxygen, phosphorous, sulfur, silicon, and/or halo).

By “heteroaromatic” is meant a subset of heterocyclyl groups, as defined herein, which are aromatic, i.e., they contain 4n+2 pi electrons within the mono- or multicyclic ring system; or a subset of aromatic groups, as defined herein, having one or more heteroatoms, i.e., they contain one or more heteroatoms within the mono- or multicyclic ring system.

By “heteroaryl” is meant an aryl group including at least one heteroatom to six heteroatoms, such as one to four heteroatoms, which can be selected from, but not limited to, oxygen, nitrogen, sulfur, silicon, boron, selenium, phosphorous, and oxidized forms thereof within the ring. Such heteroaryl groups can have a single ring or multiple condensed rings, where the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, aromatic, other functional groups, or any combination thereof. An exemplary heteroaryl includes a subset of heterocyclyl groups, as defined herein, which are aromatic, i.e., they contain 4n+2 pi electrons within the mono- or multicyclic ring system.

By “heteroarylene” is meant a multivalent (e.g., bivalent, trivalent, tetravalent, etc.) form of a heteroaryl group, as described herein. The heteroarylene group can be branched or unbranched. The heteroarylene group can also be substituted or unsubstituted. For example, the heteroarylene group can be substituted with one or more substitution groups, as described herein for alkyl or aryl.

By “heteroatom” is meant an atom other than carbon, such as oxygen, nitrogen, sulfur, silicon, boron, selenium, or phosphorous. In particular disclosed embodiments, such as when valency constraints do not permit, a heteroatom does not include a halogen atom.

By “heterocyclyl” or “heterocyclic” is meant a 5—, 6—, or 7-membered ring, unless otherwise specified, containing one, two, three, or four non-carbon heteroatoms (e.g., independently selected from the group consisting of nitrogen, oxygen, phosphorous, sulfur, or halo). The 5-membered ring has zero to two double bonds and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include thiiranyl, thietanyl, tetrahydrothienyl, thianyl, thiepanyl, aziridinyl, azetidinyl, pyrrolidinyl, piperidinyl, azepanyl, pyrrolyl, pyrrolinyl, pyrazolyl, pyrazolinyl, pyrazolidinyl, imidazolyl, imidazolinyl, imidazolidinyl, pyridyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl, and the like. The heterocyclyl group can also be substituted or unsubstituted. For example, the heterocyclyl group can be substituted with one or more groups including those described herein for alkyl or aryl.

By “hydroxyl” is meant-OH.

By “hydroxyalkyl” is meant an optionally substituted alkyl, as defined herein, having one or more hydroxyl substituents.

By “imino” is meant —NR— or —N<, in which R can be H, optionally substituted aliphatic, alkyl, heteroaliphatic, heteroalkyl, aromatic, or aryl.

By “isocyanato” is meant —N═C═O.

By “isothiocyanato” is meant —N═C═S.

By “maleimido,” “maleimidyl,” or “maleimide” is meant

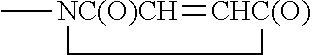

or a compound including such a group. Optional substitutions for hydrogen atoms can include a halo group, a sulfo group, and the like (e.g., any optional substitutions described herein for alkyl).

By “nitrilo” is meant —N< or —N═ or N≡.

By “nitro” is meant —NO₂.

By “oxiranyl,” “epoxy,” or “epoxide” is meant

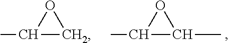

or a compound including such a group, in which one or more hydrogen atoms can be optionally substituted with another functional group. An example of an epoxy can include RCHOCH2, in which R is selected from aliphatic, heteroaliphatic, aromatic, as defined herein, or any combination thereof. Another example of an epoxy can include RCHOCHR′, in which each of R and R′ is, independently, selected from aliphatic, heteroaliphatic, aromatic, as defined herein, or any combination thereof; or R and R′, taken together with the carbon atom to which each are attached, form a cycloalkyl group, as defined herein. Optional substitutions for hydrogen atoms can include an alkyl group and the like.

By “oxy” is meant —O—.

By “sulfonyl” or “sulfo” is meant —SO₂—.

By “thioalkoxy” is meant-S-Ak, where Ak is an optionally substituted aliphatic group or an optionally substituted alkyl group, as described herein. The thioalkoxy group can be substituted or unsubstituted. For example, the thioalkoxy group can be substituted with one or more substitution groups, as described herein for alkyl. Exemplary unsubstituted thioalkoxy groups include C₁₋₃, C₁₋₆, C₁₋₁₂, C₁₋₁₆, C₁₋₁₈, C₁₋₂₀, or C₁₋₂₄ thioalkoxy groups.

By “thioaryloxy” is meant —S—Ar, where Ar is an optionally substituted aromatic group or optionally substituted aryl group, as described herein. The thioaryloxy group can be substituted or unsubstituted. For example, the thioaryloxy group can be substituted with one or more substitution groups, as described herein for alkyl or aryl. Exemplary unsubstituted thioaryloxy groups include C₄₋₁₈, C₄₋₁₄, C₄₋₁₂, C₄₋₁₀, C₄₋₁₈, C₆₋₁₄, C₆₋₁₂, or C₆₋₁₀ thioaryloxy groups.

By “thiol” is meant —SH.

By “salt” is meant an ionic form of a compound or structure (e.g., any formulas, compounds, or compositions described herein), which includes a cation or anion compound to form an electrically neutral compound or structure. Salts (e.g., simple salts having binary compounds, double salts, triple salts, etc.) are well known in the art. For example, salts are described in Berge S M et al., “Pharmaceutical salts,” J. Pharm. Sci. 1977 January; 66 (1): 1-19; International Union of Pure and Applied Chemistry, “Nomenclature of Inorganic Chemistry,” Butterworth & Co. (Publishers) Ltd., London, England, 1971 (2nd ed.); and in “Handbook of Pharmaceutical Salts: Properties, Selection, and Use,” Wiley-VCH, April 2011 (2nd rev. ed., eds. P. H. Stahl and C. G. Wermuth). The salts can be prepared in situ during the final isolation and purification of the compounds of the document or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecyl sulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

By “pharmaceutically acceptable salt” is meant a salt that is, within the scope of sound medical judgment, suitable for use in contact with the tissues of humans and animals without undue toxicity, irritation, allergic response and the like and are commensurate with a reasonable benefit/risk ratio.

By “pharmaceutically acceptable excipient” is meant any ingredient other than a compound or structure (e.g., any formulas, compounds, or compositions described herein) and having the properties of being non-toxic and non-inflammatory in a subject. Exemplary, non-limiting excipients include adjuvants, antiadherents, antioxidants, binders, carriers, coatings, compression aids, diluents, disintegrants, dispersing agents, dyes (colors), emollients, emulsifiers, fillers (diluents), film formers or coatings, flavors, fragrances, glidants (flow enhancers), isotonic carriers, lubricants, preservatives, printing inks, solvents, sorbents, stabilizers, suspensing or dispersing agents, surfactants, sweeteners, waters of hydration, or wetting agents. Any of the excipients can be selected from those approved, for example, by the United States Food and Drug Administration or other governmental agency as being acceptable for use in humans or domestic animals. Exemplary excipients include, but are not limited to alcohol, butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, glycerol, hydroxypropyl cellulose, hydroxypropyl methylcellulose, lactated Ringer's solution, lactose, magnesium stearate, maltitol, maltose, mannitol, methionine, methylcellulose, methyl paraben, microcrystalline cellulose, polyethylene glycol, polyol, polyvinyl pyrrolidone, povidone, pregelatinized starch, propyl paraben, retinyl palmitate, Ringer's solution, shellac, silicon dioxide, sodium carboxymethyl cellulose, sodium chloride injection, sodium citrate, sodium starch glycolate, sorbitol, starch (corn), stearic acid, stearic acid, sucrose, talc, titanium dioxide, vegetable oil, vitamin A, vitamin E, vitamin C, water, and xylitol.

As used herein, “administration,” “administering,” and variants thereof refers to introducing a compound or a composition into a subject and includes concurrent and sequential introduction of a compound or a composition. “Administration” can refer, e.g., to therapeutic, pharmacokinetic, diagnostic, research, placebo, and experimental methods. “Administration” also encompasses in vitro and ex vivo treatments. The introduction of a compound or a composition into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the compound or the composition to perform its intended function.

As used herein, the terms “subject,” “individual,” and “patient” are used interchangeably herein and refer to any animal subject for whom diagnosis, treatment, or therapy is desired, particularly humans. In some embodiments, the subject is a mammal (e.g., a human subject). In some embodiments, the subject is a non-human mammal (e.g., mouse, rat, guinea pig, dog, cat, horse, cow, pig, rabbit, sheep, or non-human primate, such as a monkey, chimpanzee, or baboon).

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the terms “therapeutically effective amount” and “effective amount” are used interchangeably to refer to an amount of a compound or a composition that is sufficient to provide the intended benefit (e.g. prevention, prophylaxis, delay of onset of symptoms, or amelioration of symptoms of a disease). In prophylactic or preventative applications, an effective amount can be administered to a subject susceptible to, or otherwise at risk of developing a disease, disorder or condition to eliminate or reduce the risk, lessen the severity, or delay the onset of the disease, disorder or condition, including a biochemical, histologic and/or behavioral symptoms of the disease, disorder or condition, its complications, and intermediate pathological phenotypes.

As used herein, the terms “treat,” “treating,” and/or “treatment” include abrogating, substantially inhibiting, slowing or reversing the progression of a disorder, disease or condition, substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition); (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s). Beneficial or desired clinical results, such as pharmacologic and/or physiologic effects include, but are not limited to, preventing the disease, disorder or condition from occurring in a subject predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (e.g., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Other features and advantages of the present document will be apparent from the following detailed description, the figures, and the claims.

### BRIEF DESCRIPTION OF THE DRAWINGS

The following drawings illustrate certain embodiments of the features and advantages of this document. These embodiments are not intended to limit the scope of the appended claims in any manner. Like reference symbols in the drawings indicate like elements.

FIG. 1A-1C shows schematics of (A) a non-limiting compound having a specificity group (SG) and a reactive moiety (RM); (B) a non-limiting interaction of a compound with a target having a specific target site and a reactive site; and (C) a non-limiting interaction of a compound with a multimeric target having a specific target site and a reactive site.

FIG. 2A-2B shows schematics of (A) another non-limiting compound having a specificity group (SG, including SG1 and SG2) and a reactive moiety (RM, including RM1 and RM2); and (B) a non-limiting interaction of a compound with two targets that each have a specific target site and a reactive site.

FIG. 3A-3B shows structures of non-limiting compounds, which include IC9 and SH001-SH021. Abbreviations are as follows: benzyl (Bn), methyl (Me), tert-butyl (tBu), and benzoyl (Bz).

FIG. 4A-4D shows structures of non-limiting compounds, which include SH022-SH053. Abbreviations are as follows: phenyl (Ph), benzyl (Bn), and methyl (Me).

FIG. 5 shows non-limiting results of cysteine druggability mapping (CDM) for cell models H3122 and H2228.

FIG. 6 shows a non-limiting schematic for performing a small molecule screen with a library of compounds.

FIG. 7A-7B shows non-limiting results of a small molecule screen that identified IC9 as dimerizing EML4.

FIG. 8A-8B shows non-limiting results of the interaction of EML4 domains with IC9.

FIG. 9A-9C shows non-limiting results of the interaction of cysteines in EML4 with IC9.

FIG. 10A-10B shows non-limiting results of the interaction of cysteines in EML4 with IC9.

FIG. 11 shows a non-limiting schematic of proposed 1C9 action.

FIG. 12A-12B shows non-limiting results of determining off-target binding and dimerization of 1C9 by gel-based proteomic analysis.

FIG. 13A-13B shows non-limiting results of determining off-target binding of 1C9.

FIG. 14A-14C shows non-limiting results of screening a library of compounds exhibiting ALK dimerization.

FIG. 15A-15C shows non-limiting results of cell viability assays with 1C9 and SH031.

FIG. 16 shows a non-limiting schematic of a proteolysis targeting chimera (PROTAC) including 1C9 and a cereblon ligand.

FIG. 17A-17B shows (A) non-limiting results of 1C9 dimerizing a protein including an EML4 coiled-coil domain, cysteine, and GFP and (B) non-limiting schematics of dimer, trimer, and tetramer configurations that could be implemented.

FIG. 18 shows a non-limiting schematic of the gene fusion of EML4 and ALK, which results in constitutive activation of ALK signaling and drives cancer.

FIG. 19A-19C shows non-limiting schematics of (A) targetable cysteine groups, (B) a cysteine druggability mapping (CDM) of cell model H3122, and (C) non-limiting examples of inhibitor drugs and targets.

FIG. 20A-20C shows non-limiting schematics for performing a small molecule screen with (A) pooled compounds from a library and (B) individual compounds within a pool to provide (C) screening identification of possible compounds.

FIG. 21A-21B shows non-limiting results that (A) EML4 dimerization occurs in the presence of at least one cysteine and (B) 1C9 dimerizes ALK resistant mutant cells.

FIG. 22 shows non-limiting results that EML4-ALK crosslinking leads to degradation.

FIG. 23A-23B shows non-limiting results of live-cell imaging that was conducted at the single-cell level within 24 hours of treatment.

FIG. 24A-24B shows non-limiting results of determining off-target binding and dimerization of 1C9 by gel-based proteomic analysis.

FIG. 25A-25B shows non-limiting results of an in vitro dimerization assay with a library of compounds.

FIG. 26 shows non-limiting results of cell viability assays in the presence of SH043.

FIG. 27 shows a non-limiting schematic of proposed 1C9 action.

### DETAILED DESCRIPTION

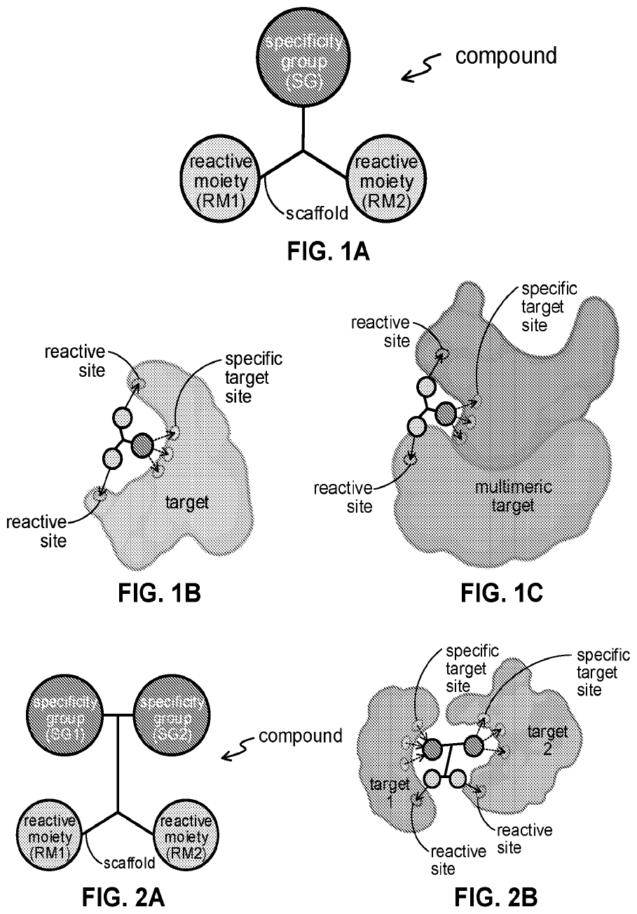

The present document relates to chemical compounds, as well as methods of making and using such chemical compounds. The chemical compound can include at least one specificity group and at least one reactive moiety, which are described herein. The specificity group can be used to engage with a specific target site of the target, and the reactive moiety can be used to covalently bind the chemical compound to a reactive site of the target. FIG. 1A-1C and FIG. 2A-2B provide non-limiting schematics of chemical compounds having a specificity group (SG) and a reactive moiety (RM1, RM2), as well as possible interactions between a chemical compound and its target.

As seen in FIG. 1A, the compound can include a scaffold disposed between a specificity group (SG) and the at least two reactive moieties (RM1 and RM2). In the presence of a target (e.g., as seen in FIG. 1B), the specificity group of the chemical compound (SG) binds to one or more specific target sites of the target. In addition, the reactive moieties of the chemical compound (e.g., here RM1 and RM2) reacts with respective reactive sites of the target, thereby forming one or more covalent bonds. The scaffold can be optimized to provide the appropriate distance and/or arrangement between the reactive sites and specific target sites. In addition, the functional groups employed for the specificity groups and the reactive moieties can be selected to provide desired interactions and/or reactions.

Multimeric targets can also be employed. As seen in FIG. 1C, the specificity group of the chemical compound (SG) binds to one or more specific target sites of the multimeric target (e.g., one portion of the multimeric target). In addition, the reactive moieties of the chemical compound (e.g., here RM1 and RM2) reacts with respective reactive sites of the target, thereby forming one or more covalent bonds (e.g., within the multimeric target). Optionally, covalent bonds are formed within a single portion of the multimeric target. In some embodiments, covalent bonds are formed between differing portions of the multimeric target.

Where FIG. 1A shows two reactive moieties, more or less reactive moieties can be used for the compound. For instance, a plurality of specificity groups and reactive moieties can be employed. As seen in FIG. 2A, the compound can include a scaffold disposed between two specificity groups (SG1 and SG2) and two reactive moieties (RM1 and RM2). Furthermore, two differing targets may be targeted.

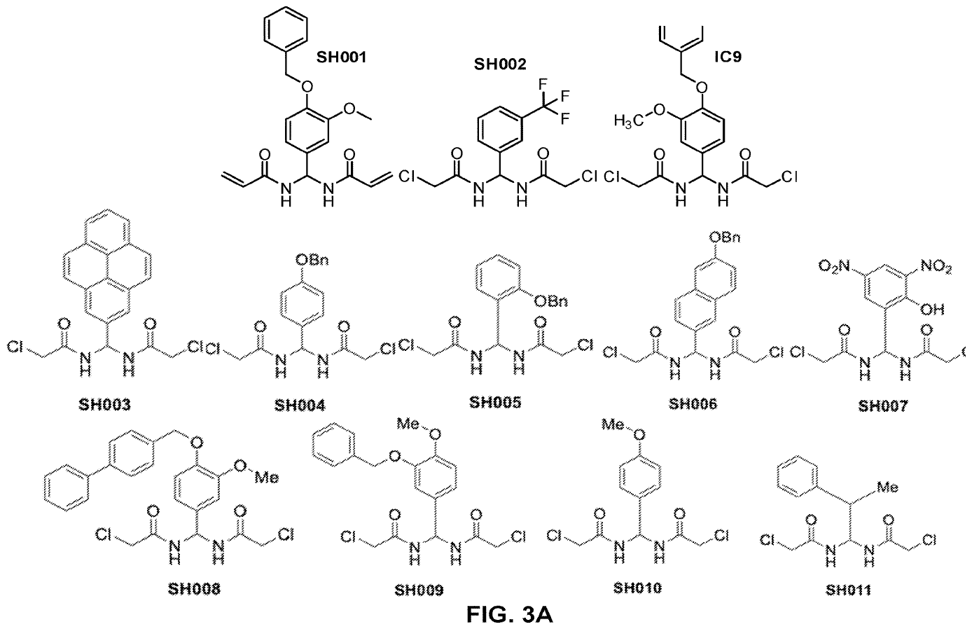

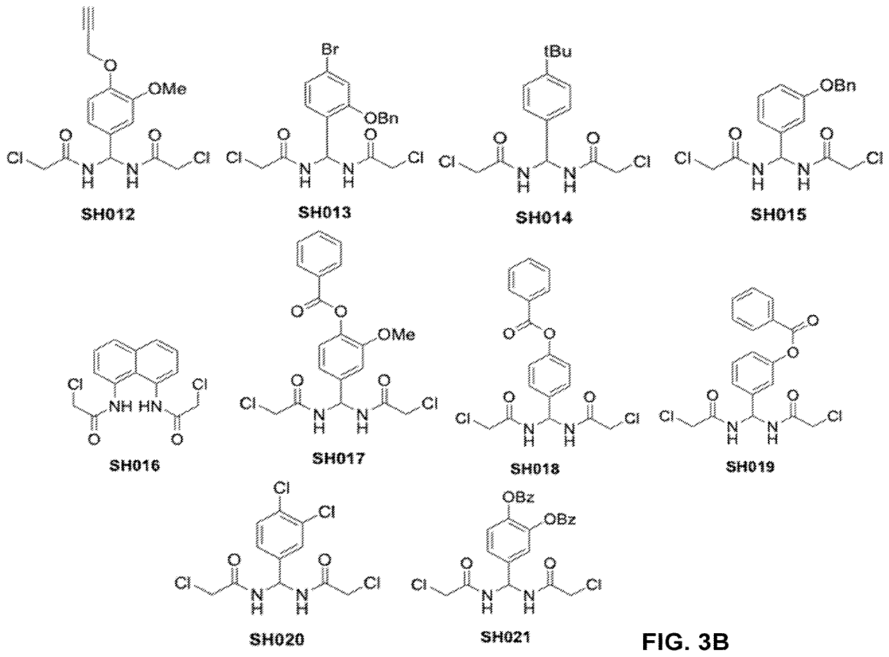

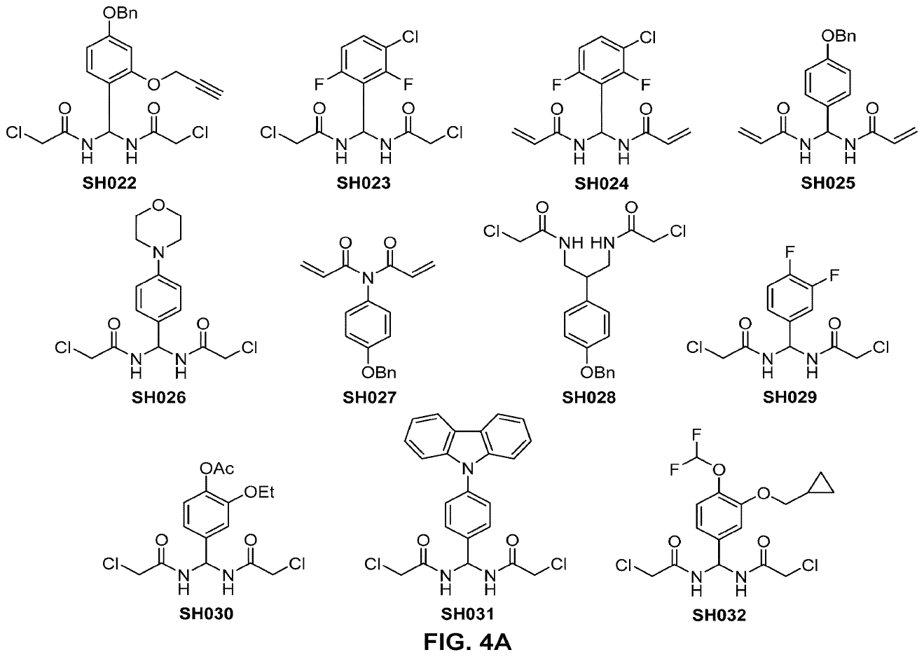

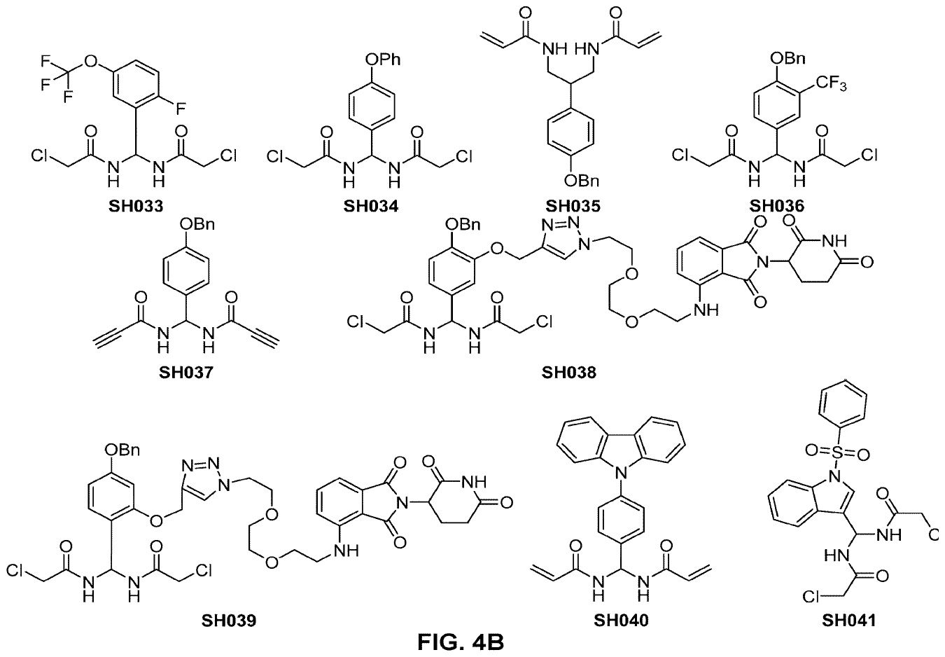

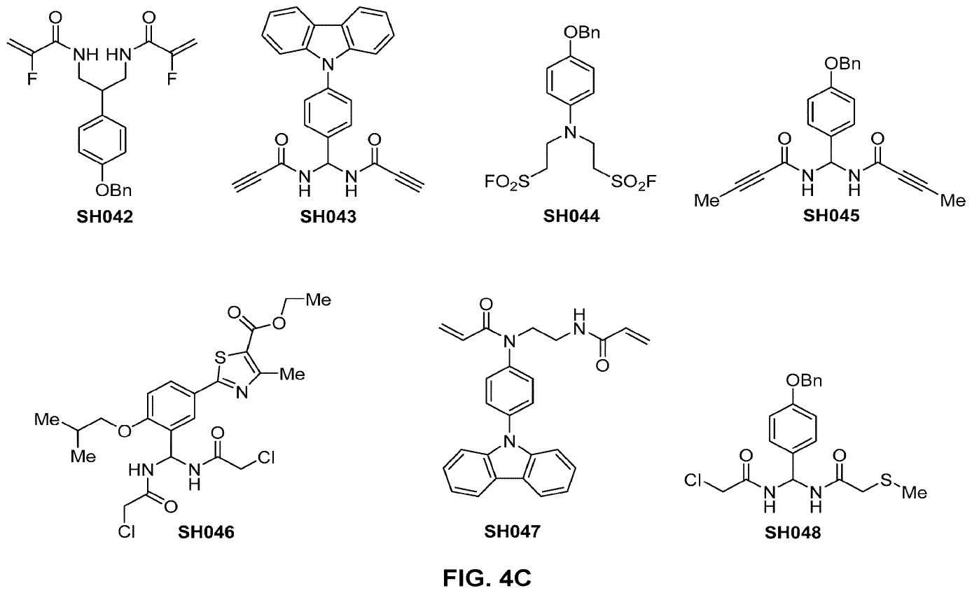

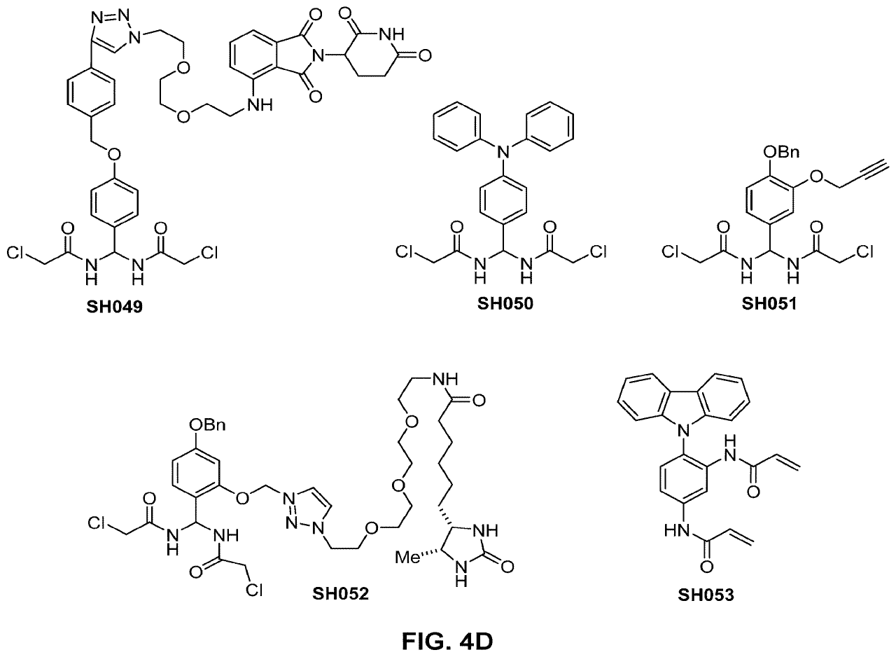

As seen in FIG. 2B, the specificity groups of the chemical compound (SG1 and SG2) binds to one or more specific target sites of target 1 and target 2. In addition, the reactive moieties of the chemical compound (e.g., RM1 and RM2) reacts with respective reactive sites of target 1 and target 2, thereby forming one or more covalent bonds (e.g., between two or more differing targets). FIG. 3A-3B and FIG. 4A-4D provide non-limiting structures of chemical compounds (e.g., compounds SH001 to SH053).

In some non-limiting instances, the specific target site can include a first region of the target, and the reactive site can include a second region of the target. In one non-limiting example, these first and second regions can be different. For example, the first region for the specific target site can include a coiled-coil region, and the chemical compound can include a specificity group that specifically engages with the coiled-coil region. In one instance, the specificity group can include an aromatic group that non-covalent interacts with the coiled-coil region. Furthermore, when the reactive site in the target includes a druggable cysteine group, then the chemical compound can include a reactive moiety that can react with a cysteine (e.g., a thiol group of the cysteine). In one instance, the reactive moiety can include a leaving group that favorably reacts with cysteine to form a covalent bond. Such a compound can form a class of covalent cysteine-reactive inhibitors.

One non-limiting target for such inhibitors can include the EML4-ALK protein, which provides a constitutively active ALK kinase domain. As discussed herein, the chemical compound can be used to inhibit the kinase activity of EML4-ALK oncogene. Without wishing to be limited by mechanism, such compounds can be used to induce multimerization of the EML4-ALK protein and to overcome ALK resistance. Additional details are provided herein, e.g., in the Examples.

Using the chemical compounds described herein, any useful method can be performed. Such methods can include engaging or targeting one or more targets by providing a chemical compound. Yet other methods can include identifying one or more targets that react with the one or more chemical compounds. Depending on the target, such compounds can be used to mitigate or modulate cellular pathways including that target. In one instance, the chemical compound can be used to treat a disease associated with a target that can be modulated by that chemical compound. Modulation can include increasing or decreasing activity, binding, or other interaction that is associated with the target (e.g., directly or indirectly associated with the target, such as downstream or upstream effects in a pathway including the target). Such chemical compounds may be provided in libraries, and methods of employing such libraries are also encompassed by the present document.

### Compounds

The present document provides compounds (e.g., for targeting or engaging one or more targets).

In some embodiments, the compound includes a structure of Formula (I):

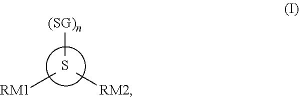

wherein:
   S includes a scaffold (e.g., a linking moiety, including a multivalent linker);
   each SG includes, independently, a specificity group configured to interact with a respective specific target site of the one or more targets;
   each of RM1 and RM2 includes, independently, a reactive moiety configured to react with a respective reactive site of the one or more targets to form covalent bonds; and
   n is an integer of 1 or more (e.g., 1, 2, or more).

In some embodiments, the compound includes a structure of Formula (Ia):

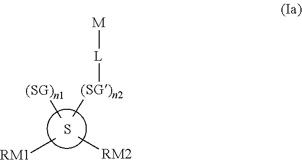

or a pharmaceutically acceptable salt thereof;
wherein:
   S includes a scaffold (e.g., a linking moiety, including a multivalent linker);
   each of SG and SG′ includes, independently, a specificity group configured to interact with a respective specific target site of the one or more targets;
   each L includes, independently, a linker (e.g., a covalent bond, oxy, carbonyl, imino, nitrilo, an atom, an optionally substituted alkylene group, an optionally substituted alkyleneoxy group, an optionally substituted heteroalkylene group, an optionally substituted arylene group, or an optionally substituted heteroarylene group, which can further optionally include a click chemistry signature, as well as any described herein);
   each of RM1 and RM2 includes, independently, a reactive moiety configured to react with a respective reactive site of the one or more targets to form covalent bonds;
   each M includes, independently, a ligand (e.g., a binding ligand, a targeting ligand, and the like, such as an E3 ubiquitin ligase (E3) ligand);
   n1 is an integer of 0, 1, or 2;
   n2 is an integer of 0, 1, or 2; and
   n1+n2 is greater than or equal to 1.

In some embodiments, the compound includes a structure of Formula (II):

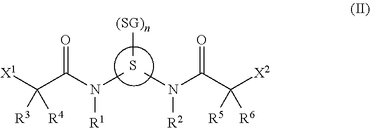

or a pharmaceutically acceptable salt thereof;
wherein:
   S includes a scaffold (e.g., a trivalent or a tetravalent linker);
   each SG includes, independently, a specificity group configured to interact with a respective specific target site of one or more targets (e.g., includes one or more aromatic groups; or one or more other targeting compounds);
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n is an integer of 1 or more (e.g., 1, 2, or more).

In some embodiments (e.g., for a compound including a structure of Formula (II)), the compound is not 2-chloro-N-[[(2-chloroacetyl)amino]-(3-methoxy-4-phenylmethoxyphenyl)methyl]acetamide.

In some embodiments, S and SG, when taken together, is not phenyl that is substituted with both methoxy in the meta-position and phenylmethoxy in the para position.

In some embodiments, the compound includes a structure having Formula (II):

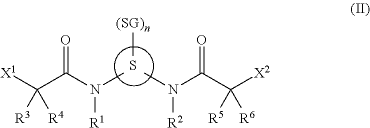

or a pharmaceutically acceptable salt thereof;
wherein:
   S includes a scaffold (e.g., a trivalent or tetravalent linker);
   each SG includes, independently, a specificity group configured to interact with a respective specific target site of one or more targets (e.g., includes one or more aromatic groups; or one or more other targeting compounds);
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶ when taken together with the carbon atom to which each are attached, forms an optionally substituted alkynyl group; and
   n is an integer of 1 or more (e.g., 1, 2, or more), and
   wherein at least one of S, SG, R¹, R², R³, R⁴, R⁵, or R⁶ includes an E3 ubiquitin ligase (E3) ligand.

One or more compounds may be present in a library. In some embodiments, the compound (e.g., at least one of a plurality of test compounds in a library) includes a structure having Formula (II):

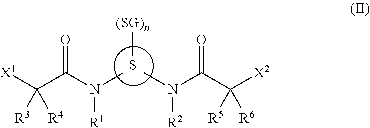

or a pharmaceutically acceptable salt thereof;
wherein:
   S includes a scaffold (e.g., a trivalent or tetravalent linker);
   each SG includes, independently, a specificity group configured to interact with a respective specific target site of one or more targets (e.g., includes one or more aromatic groups; or one or more other targeting compounds);
   each of R¹, R², R³, R⁴, R⁵, and R⁶ is, independently, H, halo, optionally substituted C_{1 12} aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n is an integer of 1 or more (e.g., 1, 2, or more), and
   wherein the compound is not 2-chloro-N-[(2-chloroacetyl)amino]-(3-methoxy-4-phenylmethoxyphenyl)methyl|acetamide; and
   optionally wherein at least one of S, SG, R¹, R², R³, R⁴, R⁵, or R⁶ includes an E3 ubiquitin ligase (E3) ligand.

In some embodiments, the compound includes a structure having Formula (IIa):

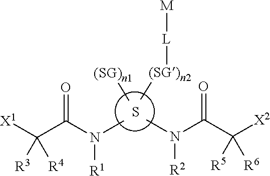

or a pharmaceutically acceptable salt thereof;
wherein:
   S includes a scaffold (e.g., a linking moiety, including a multivalent linker);
   each of SG and SG′ includes, independently, a specificity group configured to interact with a respective specific target site of the one or more targets;
   each L includes, independently, a linker (e.g., a covalent bond, oxy, carbonyl, imino, nitrilo, an atom, an optionally substituted alkylene group, an optionally substituted alkyleneoxy group, an optionally substituted heteroalkylene group, an optionally substituted arylene group, or an optionally substituted heteroarylene group, which can further optionally include a click chemistry signature, as well as any described herein);
   each M includes, independently, a ligand (e.g., a binding ligand, a targeting ligand, and the like, such as an E3 ubiquitin ligase (E3) ligand);
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
   n1 is an integer of 0, 1, or 2;
   n2 is an integer of 0, 1, or 2; and
   n1+n2 is greater than or equal to 1.

In some embodiments, the compound includes a structure having any one of the following formulas (1)-(16):

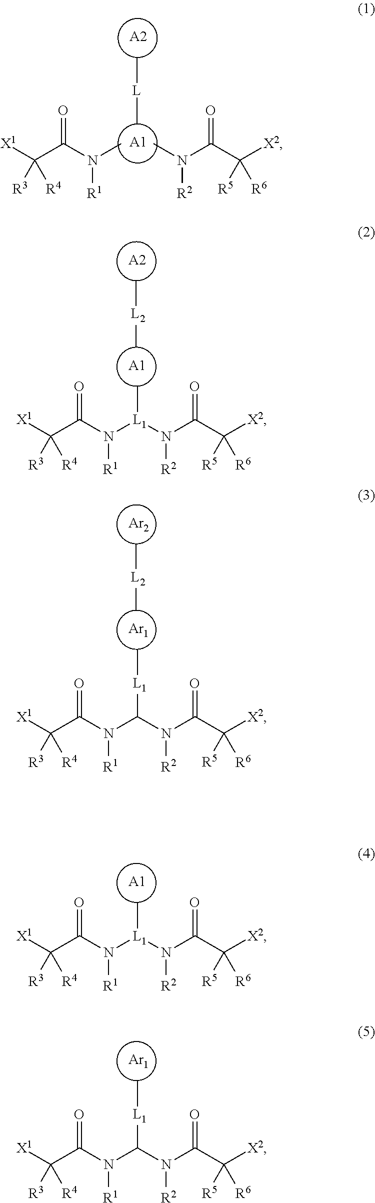

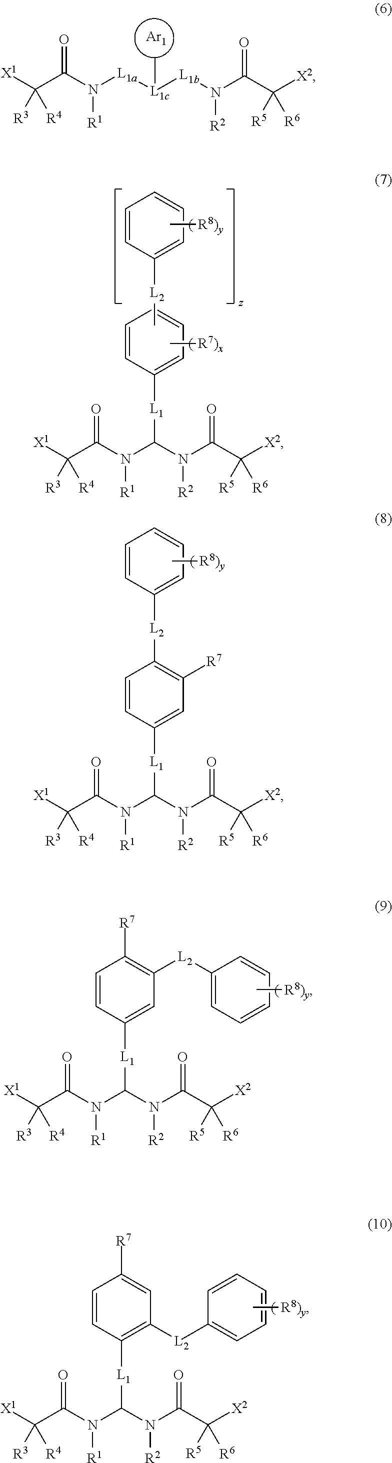

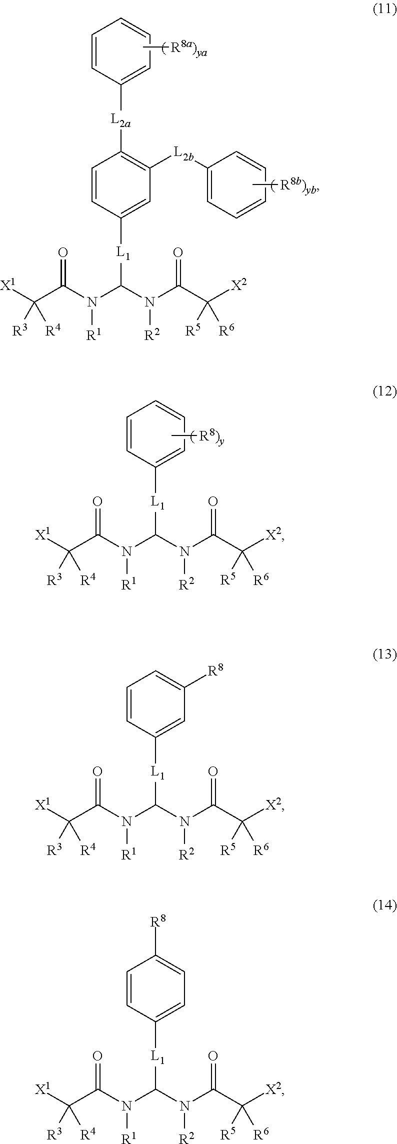

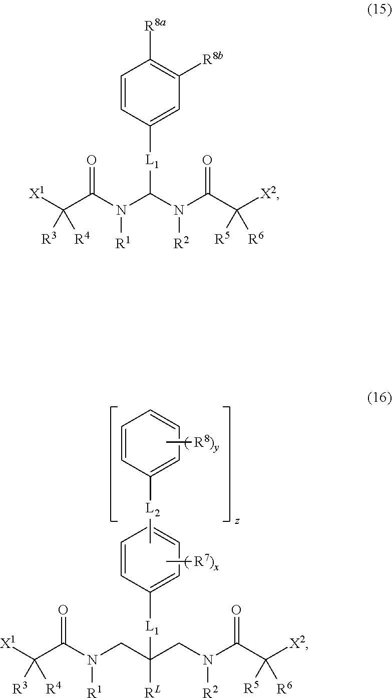

or a pharmaceutically acceptable salt thereof;
wherein:
   each of A1, A2, Ar₁, and Ar₂, includes, independently, one or more aromatic groups (e.g., any described herein, including one or more heteroaromatic groups);
   each of L, L₁, L₁ₐ, L_{1b}, L_{1c}, L₂, L₂ₐ, and L_{2b} includes, independently, a linker (e.g., a covalent bond, a bivalent linker, a trivalent linker, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group, which can further optionally include a click chemistry signature, as well as any described herein);
   each of R¹, R², R³, R⁴, R⁵, R⁶, and R^{L} is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
   each of R⁷, R⁸, R^{8a}, and R^{8b} is, independently, H, optionally substituted C₁₋₁₂ aliphatic (e.g., alkyl, cycloalkyl, haloalkyl, hydroxyalkyl, alkenyl, alkynyl), optionally substituted C₁₋₁₂ heteroaliphatic (e.g., alkoxy, haloalkoxy, alkenyloxy, alkynyloxy, alkanoyl, alkanoyloxy), optionally substituted aromatic (e.g., aryl, heteroaryl), optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano;
   x is an integer of 0-4 (e.g., an integer of 1-4);
   each of y, ya, and yb is, independently, an integer of 0-5 (e.g., an integer of 1-5); and
   z is an integer of 0-4 (e.g., an integer of 1-4).

In some embodiments, x is an integer greater than 0. In some embodiments, at least one of y, ya, and yb is an integer greater than 0. In some embodiments, z is an integer greater than 0.

In some embodiments, the compound includes a structure having any one of the following formulas (17)-(25):

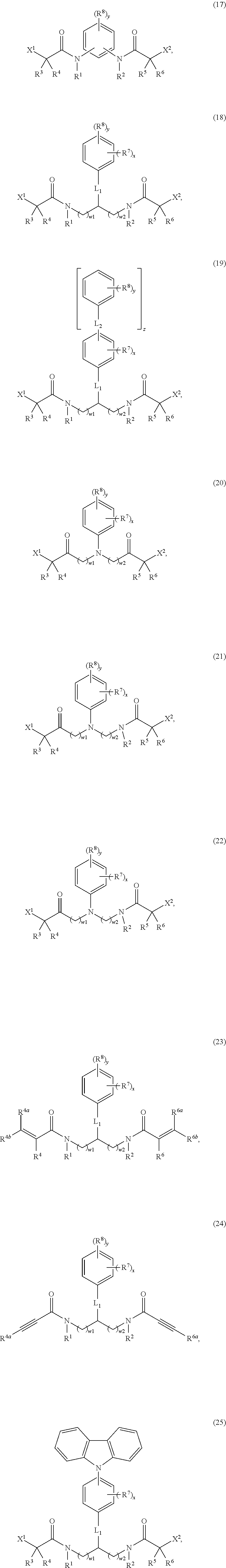

or a pharmaceutically acceptable salt thereof;
wherein:
   each of L₁ and L₂ includes, independently, a linker (e.g., a covalent bond, a bivalent linker, a trivalent linker, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group, which can further optionally include a click chemistry signature, as well as any described herein);
   each of R¹, R², R³, R⁴, R^{4a}, R^{4b}, R⁵, R⁶, R^{6a}, and R^{6b} is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
   each of R⁷ and R⁸ is, independently, H, optionally substituted C₁₋₁₂ aliphatic (e.g., alkyl, cycloalkyl, haloalkyl, hydroxyalkyl, alkenyl, alkynyl), optionally substituted C₁₋₁₂ heteroaliphatic (e.g., alkoxy, haloalkoxy, alkenyloxy, alkynyloxy, alkanoyl, alkanoyloxy), optionally substituted aromatic (e.g., aryl, heteroaryl), optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano;
   each of w1 and w2 is, independently, an integer of 0-2;
   x is an integer of 0-4 (e.g., an integer of 1-4);
   each of y, ya, and yb is, independently, an integer of 0-5 (e.g., an integer of 1-5); and
   z is an integer of 0-4 (e.g., an integer of 1-4).

In some embodiments, at least one of w1 and w2 is an integer greater than 0. In some embodiments, w1 and w2 are both 0, or at least one of w1 and w2 is 0. In some embodiments, x is an integer greater than 0. In some embodiments, at least one of y, ya, and yb is an integer greater than 0. In some embodiments, z is an integer greater than 0.

In some embodiments, the compound includes a structure having Formula (III):

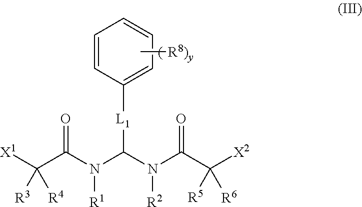

or a pharmaceutically acceptable salt thereof;
wherein:
   L₁ includes a linker (e.g., a covalent bond, a bivalent linker, a trivalent linker, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group, which can further optionally include a click chemistry signature, as well as any described herein);
   each of R¹, R², R³, R⁴, R⁵, and R⁶ is, independently, H, halo, optionally substituted C₁₋₁₂ aliphatic, or optionally substituted C₁₋₁₂ heteroaliphatic;
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
   each of R⁸ is, independently, H, optionally substituted aliphatic (e.g., alkyl, cycloalkyl, haloalkyl, hydroxyalkyl, alkenyl, alkynyl), optionally substituted heteroaliphatic (e.g., alkoxy, haloalkoxy, alkenyloxy, alkynyloxy, alkanoyl, alkanoyloxy), optionally substituted aromatic (e.g., aryl, heteroaryl), optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
   y is an integer of 1-5.

In some embodiments, the compound includes a structure having Formula (IV) or (V):

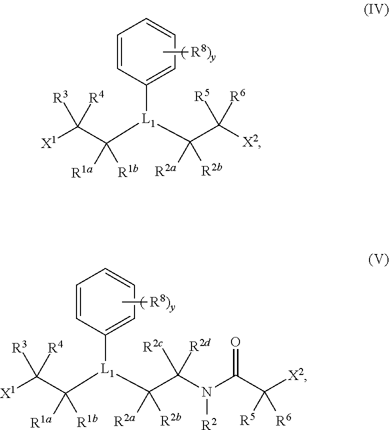

or a pharmaceutically acceptable salt thereof;
wherein:
   L₁ includes a linker (e.g., a covalent bond, a bivalent linker, a trivalent linker, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group, which can further optionally include a click chemistry signature, as well as any described herein);
   each of R^{1a}, R^{1b}, R², R^{2a}, R^{2b}, R^{2c}, R^{2d}, R³, R⁴, R⁵, and R⁶ is, independently, H, halo, optionally substituted C₁₋₁₂ aliphatic, or optionally substituted C₁₋₁₂ heteroaliphatic, or wherein R^{1a} and R^{1b}, when taken together, forms an oxo (e.g., ═O), or wherein R^{2a} and R², when taken together, forms an oxo (e.g., ═O);
   each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
   each of R⁸ is, independently, H, optionally substituted aliphatic (e.g., alkyl, cycloalkyl, haloalkyl, hydroxyalkyl, alkenyl, alkynyl), optionally substituted heteroaliphatic (e.g., alkoxy, haloalkoxy, alkenyloxy, alkynyloxy, alkanoyl, alkanoyloxy), optionally substituted aromatic (e.g., aryl, heteroaryl), optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
   y is an integer of 1-5.

In any embodiment herein, the compound (e.g., any herein, such as a compound of having any one of Formulas (I), (Ia), (II), (IIa), (III), (IV), (V), and (1)-(25)) includes a linker (e.g., any described herein) disposed between at least one of the S, SG, R¹, R², R³, R⁴, R⁵, or R⁶ and the E3 ligand.

In any embodiment herein, the specificity group, SG, and/or SG′, if present, includes an aromatic group (e.g., which can also include a heteroaromatic group).

In any embodiment herein, the aromatic group includes an optionally substituted aryl, phenyl, benzyl, biphenyl, naphthyl, pyrenyl, indenyl, fluorenyl, phenalenyl, phenanthryl, anthryl, triphenylenyl, tetracenyl, acenaphthenyl, carbazolyl (dibenzopyrrolyl), dibenzofuranyl, quinolinyl, isoquinolinyl, indolyl, benzimidazolyl, thiazolyl, or the like.

In any embodiment herein, the specificity group, SG, and/or SG′, if present, independently includes optionally substituted phenyl, optionally substituted benzyl, or optionally substituted pyrenyl.

In any embodiment herein, the specificity group, the scaffold, SG, SG′, and/or S, if present, is independently substituted with one or more of the following groups: halo (e.g., —X), hydroxyl, nitro, optionally substituted alkyl (e.g., —Ak), optionally substituted cycloalkyl, optionally substituted haloalkyl, optionally substituted haloalkoxy, optionally substituted hydroxyalkyl, optionally substituted alkoxy (e.g., —O-Ak), optionally substituted alkenyloxy, optionally substituted alkynyloxy, optionally substituted alkanoyl (e.g., —C(O)—Ak), optionally substituted alkanoyloxy (e.g., —O—C(O)—Ak), optionally substituted aromatic, optionally substituted aryl, optionally substituted aralkyl (e.g., —Ak-Ar), optionally substituted alkaryl (e.g., —Ar-Ak), optionally substituted aryloxy (e.g., —O—Ar), optionally substituted aralkoxy (e.g., —O-Ak-Ar), optionally substituted aralkanoyl (e.g., —C(O)-Ak-Ar), optionally substituted aralkanoyloxy (e.g., —O—C(O)-Ak-Ar), optionally substituted aryloyloxy (e.g., —O—C(O)—Ar), optionally substituted aryloxycarbonyl (e.g., —C(O)—O—Ar), optionally substituted aryloyl (e.g., —C(O)—Ar), optionally substituted heteroaromatic, optionally substituted heterocyclyl, optionally substituted heteroaryl, or optionally substituted amino.

In any embodiment herein, the scaffold or S, if present, includes a monovalent, bivalent, trivalent, tetravalent, or pentavalent linker (e.g., a covalent bond, oxy, carbonyl, carbonyloxy, imino, nitrilo, sulfo, an atom, an optionally substituted aliphatic group, an optionally substituted alkylene group, an optionally substituted alkyleneoxy group, an optionally substituted heteroaliphatic group, an optionally substituted heteroalkylene group, an optionally substituted aromatic group, an optionally substituted arylene group, an optionally substituted heteroarylene group, or an optionally substituted heterocyclic group, as well as any described herein).

In any embodiment herein, the reactive moiety, the at least two reactive moieties, RM1, and/or RM2, if present, independently includes an electrophilic group or a leaving group.

In any embodiment herein, the electrophilic group or the leaving group includes optionally substituted haloacetyl (e.g., —C(O)CH₂X, where X is halo), optionally substituted haloacetylamido (e.g., —NRC(O)CH₂X, where X is halo and wherein R is H or alkyl), optionally substituted acryloyl (e.g., —C(O)—CH═CH₂), optionally substituted acrylamido (e.g., —NRC(O)—CH═CH₂, where R is H or alkyl), optionally substituted alkenyl, optionally substituted alkynyl, optionally substituted oxiranyl (epoxide radical), halo, cyano (—CN), isocyanato (—NCO), isothiocyanato (—NCS), optionally substituted alkenylsulfonyl (e.g., —SO₂-vinyl), halosulfonyl (e.g., —SO₂-halo), optionally substituted maleimidyl, optionally substituted alkoxy, optionally substituted thioalkoxy, optionally substituted thioaryloxy, thiol, and the like.

In any embodiment herein, the linker (e.g., linker L that may optionally be attached to M) includes -L^{A}-L^{B}-L^{C}-, wherein: each of L^{A}, L^{B}, and L^{C} includes, independently, a linker (e.g., a covalent bond, oxy, carbonyl, carbonyloxy, sulfo, an atom, an optionally substituted alkylene group, an optionally substituted alkyleneoxy group, an optionally substituted heteroalkylene group, an optionally substituted arylene group, or an optionally substituted heteroarylene group, which can further optionally include a click chemistry signature, as well as any described herein).

In any embodiment herein, each of L^{A} and L^{B} is, independently, a covalent bond, oxy, carbonyl, carbonyloxy, imino, nitrilo, sulfo, an atom, an optionally substituted alkylene group, an optionally substituted alkyleneoxy group, an optionally substituted heteroalkylene group, an optionally substituted arylene group, or an optionally substituted heteroarylene group.

In any embodiment herein, L^{C} includes a click chemistry signature (e.g., an optionally substituted triazolyl).

In any embodiment herein, one or more aromatic groups (e.g., one or more of A1, A2, Ar₁, and Ar₂) is substituted with one or more groups selected from optionally substituted aliphatic (e.g., alkyl, cycloalkyl, haloalkyl, hydroxyalkyl, alkenyl, alkynyl), optionally substituted heteroaliphatic (e.g., alkoxy, haloalkoxy, alkenyloxy, alkynyloxy, alkanoyl, alkanoyloxy), optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, optionally substituted alkylsulfonyl, optionally substituted arylsulfonyl, halo, hydroxyl, nitro, or cyano.

In any embodiment herein, each of R¹, R², R³, R⁴, R^{4a}, R^{4b}, R⁵, R⁶, R^{6a}, and R^{6b} is, independently, H, halo, haloalkyl, or alkyl.

In any embodiment herein, each of R^{1a}, R^{1b}, R², R^{2a}, R^{2b}, R^{2c}, R^{2d}, R³, R⁴, R^{4a}, R^{4b}, R⁵, R⁶, R^{6a}, and R^{6b} is, independently, H, halo, haloalkyl, or alkyl.

In any embodiment herein, R⁷ is H, halo, hydroxyl, nitro, cyano, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, R⁷ is H, halo, hydroxyl, nitro, optionally substituted alkyl, optionally substituted cycloalkyl, optionally substituted haloalkyl, optionally substituted haloalkoxy, optionally substituted alkoxy, optionally substituted alkenyloxy, or optionally substituted alkynyloxy.

In any embodiment herein, at least one of R⁷ and R⁸ is not H.

In any embodiment herein, each of R⁷, R⁸, R^{8a}, and R^{8b} is, independently, H, halo, hydroxyl, nitro, cyano, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, R⁸ is not H. In some embodiments, R⁸ is not halo. In some embodiments, R⁸ is not alkyl.

In any embodiment herein, R⁸, R^{8a}, and R^{8b} are not H. In some embodiments, R⁸, R^{8a}, and R^{8b} are not halo. In some embodiments, R⁸, R^{8a}, and R^{8b} are not alkyl.

In any embodiment herein, R⁸, R^{8a} (if present), and R^{8b} (if present) is, independently, H, halo, hydroxyl, nitro, cyano, optionally substituted alkyl, optionally substituted cycloalkyl, optionally substituted haloalkyl, optionally substituted haloalkoxy, optionally substituted hydroxyalkyl, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, R⁸, R^{8a} (if present), and R^{8b} (if present) is, independently, H, halo, optionally substituted alkyl, optionally substituted cycloalkyl, optionally substituted haloalkyl, optionally substituted haloalkoxy, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, optionally substituted heteroaryl, or optionally substituted amino.

In any embodiment herein, R⁸, R^{8a} (if present), and R^{8b} (if present) is, independently, halo, optionally substituted haloalkyl, optionally substituted haloalkoxy, optionally substituted alkoxy, optionally substituted alkenyloxy, optionally substituted alkynyloxy, optionally substituted alkanoyl, optionally substituted alkanoyloxy, optionally substituted aromatic, optionally substituted aryl, optionally substituted aralkyl, optionally substituted alkaryl, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heteroaromatic, optionally substituted heterocyclyl, or optionally substituted heteroaryl.

In any embodiment herein, the compound (e.g., within the scaffold or S) can include a linker, such as a monovalent, bivalent, trivalent, tetravalent, or pentavalent linker. Non-limiting examples of linkers can include any described herein. In some embodiments, the linker is a covalent bond, oxy (e.g., —O—), carbonyl (e.g., —C(O)—), carbonyloxy (e.g., —O—C(O)— or —C(O)—O—), imino (e.g., —NR— or —N<), nitrilo (e.g., —N< or —N═ or N═), sulfo (e.g., —SO₂—), an atom, optionally substituted aliphatic, optionally substituted alkylene, optionally substituted alkyleneoxy, optionally substituted heteroaliphatic, optionally substituted heteroalkylene, optionally substituted aromatic, optionally substituted arylene group, optionally substituted heteroarylene, or optionally substituted heterocyclic, as well as any described herein.

Any of the compounds herein (alone or in combination) can be provided in a composition. In some embodiments, the composition is a pharmaceutical composition. In some embodiments, the pharmaceutical composition includes a chemical compound (e.g., any described herein) and a pharmaceutically acceptable excipient. In some embodiments, the chemical compound is present in a therapeutically effective amount for a unit dosage form. Non-limiting examples of unit dosage forms include an oral dosage form (e.g., a tablet, capsule, caplet, gelcap, or syrup), a topical dosage form (e.g., a cream, gel, lotion, or ointment), or an intravenous dosage form (e.g., as a sterile solution).

Any of the compounds herein (alone or in combination) can be provided in a library. In some embodiments, the library can be encoded to provide the identity of the compound. The library can include a plurality of compounds (e.g., including one or more of any compounds herein) provided in any useful format (e.g., beads, wells, arrays, etc.).

### Methods

The present document encompasses methods of using a compound (e.g., any described herein).

A non-limiting method can include a method of targeting or engaging one or more targets with a chemical compound (e.g., any described herein). In some embodiments, the method includes: binding a specificity group (e.g., SG, such as any described herein) of the chemical compound to a specific target site, and reacting at least two reactive moieties (e.g., RM, such as RM1, RM2, or any described herein) of the chemical compound with respective reactive sites to form covalent bonds.

In some embodiments, the one or more targets includes the specific target site and at least two reactive sites. For example and without limitation, the one or more targets can include a monomeric protein, a multimeric protein, a single protein, or a plurality of proteins.

In some embodiments, the chemical compound includes a scaffold (e.g., S, such as any described herein) disposed between the specificity group and the at least two reactive moieties.

A non-limiting method can include a method of targeting or engaging one or more targets with a chemical compound, the method including: providing a chemical compound (e.g., any described herein) to the one or more targets, and incubating the chemical compound with the one or more targets under conditions configured to bind the specificity group to the specific target site and to react the at least two reactive moieties with the respective reactive site to form covalent bonds.

In some embodiments, the chemical compound includes: a specificity group (e.g., SG, such as any described herein) configured to interact with a specific target site of the one or more targets, and at least two (e.g., two, three, four, or more) reactive moieties (e.g., RM, such as RM1, RM2, or any described herein), wherein each of the at least two reactive moieties is configured to react with a respective reactive site of the one or more targets.

A non-limiting method can include a method of targeting or engaging one or more targets with a chemical compound, the method including: providing an effective amount of the chemical compound, wherein the chemical compound includes a structure having any one of Formulas (I), (Ia), (II), (IIa), (III), (IV), (V), and (1)-(25), or a pharmaceutically acceptable salt thereof. In some embodiments, the chemical compound is one or more of SH001-SH053, or a pharmaceutically acceptable salt thereof. In some embodiments, the chemical compound is one or more of SH002, SH003, SH004, SH005, SH006, SH009, SH012, SH013, SH014, SH015, SH016, SH017, SH018, SH019, SH020, SH021, SH022, SH023, SH028, SH031, SH032, SH034, SH036, SH037, SH038, SH039, SH040, SH043, or a pharmaceutically acceptable salt thereof.

A non-limiting method can include a method of identifying one or more targets, the method including: providing a chemical compound to a test sample, wherein the chemical compound includes a structure having any one of Formulas (I), (Ia), (II), (IIa), (III), (IV), (V), and (1)-(25), or a pharmaceutically acceptable salt thereof; and identifying a biological target bound to the chemical compound. In some embodiments, the chemical compound is one or more of SH001-SH053, or a pharmaceutically acceptable salt thereof. In some embodiments, the chemical compound is one or more of SH002, SH003, SH004, SH005, SH006, SH009, SH012, SH013, SH014, SH015, SH016, SH017, SH018, SH019, SH020, SH021, SH022, SH023, SH028, SH031, SH032, SH034, SH036, SH037, SH038, SH039, SH040, SH043, or a pharmaceutically acceptable salt thereof.

A non-limiting method can include a method of treating a disease (e.g., cancer), the method including: administering a therapeutically effective amount of a chemical compound to a subject in need thereof, wherein the chemical compound includes a structure having any one of Formulas (I), (Ia), (II), (IIa), (III), (IV), (V), and (1)-(25), or a pharmaceutically acceptable salt thereof. In some embodiments, the chemical compound is one or more of SH001-SH053, or a pharmaceutically acceptable salt thereof. In some embodiments, the chemical compound is one or more of SH002, SH003, SH004, SH005, SH006, SH009, SH012, SH013, SH014, SH015, SH016, SH017, SH018, SH019, SH020, SH021, SH022, SH023, SH028, SH031, SH032, SH034, SH036, SH037, SH038, SH039, SH040, SH043, or a pharmaceutically acceptable salt thereof.

In some embodiments, the disease is cancer. Non-limiting examples include non-small cell lung cancer (NSCLC), lung adenocarcinoma, lung cancer, breast cancer, and colorectal cancer. In some embodiments, the cancer is characterized by a presence of an EML4-ALK fusion gene, an EML4-ALK fusion protein, an EML4 fusion gene, an EML4 fusion protein, an ALK fusion gene, an ALK fusion protein, or a variant thereof.

In some embodiments, the non-small cell lung cancer (NSCLC) is characterized by a presence of an EML4-ALK fusion gene, an EML4-ALK fusion protein, an EML4 fusion gene, an EML4 fusion protein, an ALK fusion gene, an ALK fusion protein, or a variant thereof.

In some embodiments, the specificity group and/or the scaffold, if present, includes an E3 ubiquitin ligase (E3) ligand. In some embodiments, the compound (e.g., any described herein) includes a linker (e.g., any described herein) disposed between the specificity group and the E3 ligand and/or between the scaffold and the E3 ligand.

### Targets

The present document relates to compounds and methods for binding, targeting, or otherwise interacting within any target. In some embodiments, the target includes a protein, a peptide, a fusion protein, or a constitutively active receptor protein. Non-limiting examples of targets include, e.g., EML4-ALK, EML4, EML4 fusion protein, ALK fusion protein, PSME1, PSME2, TMX1, TXN, TJP2, PDIA6, P4HB, AGR2, PDIA3, PDIA4, PPFIAI, SMARCC2, GCLC, NPEPPS, PDCD61P, PRDX1, ERO1A, PCBP1, CTTN, PRDX2, PRDX3, RARSI, TXNDC17, RPL21, S100A2, KIF5B, or RPL18. In some embodiments, the one or more targets includes an ALK fusion protein having one or more optional mutations (e.g., 1151T-ins, L1152R/P, C1156Y, 11171T/N/S, F1174L/C/V, V1180L, L1196M/F, L1198P, G1202R/del, D1203N, S1206Y, E1210K, and/or G1269A, including combinations of any of these, such as L1196M/G1202R).

Targets can also be characterized by the presence of certain structural domains or interactions. In some embodiments, the one or more targets includes a coiled-coil domain. In some embodiments, the specific target site includes the coiled-coil domain.

Targets can also be characterized by the presence of certain functional groups (e.g., amino acids). In some embodiments, the one or more targets includes one or more cysteines. In some embodiments, at least two reactive sites includes at least two cysteines. In some embodiments, the reactive site includes the cysteine.

Targets can be present, e.g., within a cell, a culture, a tissue sample, a lysate, a sample, and the like.

In some embodiments, the method, the chemical compound, or the test compound inhibits the one or more targets. In some embodiments, the method, the chemical compound, or the test compound inhibits signaling of the one or more target (e.g., wherein the signaling includes signaling of kinase activity).

### Ligands

Any of the compounds herein can include a ligand. In some embodiments, the ligand can engage, bind, or otherwise interact with an E3 ubiquitin ligase. In turn, such a ligand can be an E3 ligand. In some embodiments, the E3 ligand is a ligand for Skp1-Cullin-F box complex (SCF^{β-TrCP}), von Hippel-Lindau (VHL), murine double minute 2 (MDM2), an inhibitor of apoptosis proteins (IAP, e.g., cIAP1, cIAP2, XIAP, or ML-IAP), or cereblon (CRBN).

Non-limiting examples of ligands include, e.g., a thalidomide-based ligand, a pomalidomide-based ligand, a 4-hydroxythalidomide-based ligand, a lenalidomide-based ligand, a VH032-based ligand, a VHL ligand 1 (VHL Al)-based ligand, a nutlin-3-based ligand, an idasanutlin-based ligand, a bestatin-based ligand, a methyl bestatin-based ligand, a LCL-161-based ligand, and the like.

The ligand can be provided in any useful manner. In some embodiments, the ligand is a substitution for or attached to one or more of S, SG, SG′, A1, A2, Ar₁, Ar₂, R⁷, R⁸, R^{8a}, and R^{8b}.

### Linkers

Linkers can be present between two components (e.g., between a click chemistry entity and a ligand, between a reactive group and ligand, etc.) or within a scaffold or S. Linkers can include a bond (e.g., a covalent bond); an optionally substituted alkylene; an optionally substituted heteroalkylene (e.g., poly(ethylene glycol), such as —(OCH₂CH₂)ₙ—, in which n is an integer of 1 to 100); an optionally substituted arylene; or an optionally substituted heteroarylene, as well as combinations thereof.

An alkylene can include a multivalent (e.g., bivalent, trivalent, tetravalent, etc.) form of an alkyl group. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups (e.g., halo, oxy, oxo, amino, and the like). A heteroalkylene can be an alkylene group containing one, two, three, or four non-carbon heteroatoms (e.g., independently selected from the group consisting of nitrogen, oxygen, phosphorous, sulfur, or halo).

The linker can include one or more chemical signatures. In one embodiment, the chemical signature includes a click-chemistry signature, which arises from reacting a click-chemistry reaction pair (e.g., any described herein). Non-limiting examples of click-chemistry signatures include a triazole, an unsaturated six-member ring, a covalent bond, and the like.

In another embodiment, the chemical signature can include a reaction signature, which arises from reacting a cross-linker reaction pair. Non-limiting examples of cross-linker reaction pairs include those for forming a covalent bond between a carboxyl group (e.g., —CO₂H) and an amino group (e.g., —NH₂); or between an imido group (e.g., maleimido or succinimido) and a thiol group (e.g., —SH); or between an epoxide group and a thiol group (e.g., —SH); or between an epoxide group and an amino group (e.g., —NH₂); or between an ester group (e.g., —CO₂R, in which R is an organic moiety, such as optionally substituted alkyl, aryl, etc.) and an amino group (e.g., —NH₂); or between an carbamido group (e.g., —NHC(O)Het, where Het is a N-containing heterocyclyl) and an amino group (e.g., —NH₂); or between a phospho group (e.g., —P(O)(OH)₂) and an amino group (e.g., —NH₂), such as 1-ethyl-3-(3-dimethylaminopropyl) carbodiimide (EDC) and dicyclohexylcarbodiimide (DCC), optionally used with N-hydroxysuccinimide (NHS) and/or N-hydroxysulfosuccinimide (sulfo-NHS). Other cross-linkers include those for forming a covalent bond between an amino group (e.g., —NH₂) and a thymine moiety, such as succinimidyl-[4-(psoralen-8-yloxy)]-butyrate (SPB); a hydroxyl group (e.g., —OH) and a sulfur-containing group (e.g., free thiol,—SH, sulfhydryl, cysteine moiety, or mercapto group), such as p-maleimidophenyl isocyanate (PMPI); between an amino group (e.g., —NH₂) and a sulfur-containing group (e.g., free thiol, —SH, sulfhydryl, cysteine moiety, or mercapto group), such as succinimidyl 4-(p-maleimidophenyl) butyrate (SMPB) and/or succinimidyl 4-(N-maleimidomethyl)cyclohexane-1-carboxylate (SMCC); between a sulfur-containing group (e.g., free thiol, —SH, sulfhydryl, cysteine moiety, or mercapto group) and a carbonyl group (e.g., an aldehyde group, such as for an oxidized glycoprotein carbohydrate), such as N-beta-maleimidopropionic acid hydrazide-trifluoroacetic acid salt (BMPH), 3-(2-pyridyldithio) propionyl hydrazide (PDPH), and/or a 3-(2-pyridyldithio) propionyl group (PDP); and between a maleimide-containing group and a sulfur-containing group (e.g., free thiol, —SH, sulfhydryl, cysteine moiety, or mercapto group). Yet other cross-linkers include those for forming a covalent bond between two or more unsaturated hydrocarbon bonds, e.g., mediated by radical polymerization, such as a reaction of forming a covalent bond between a first alkene group and a second alkene group (e.g., a reaction between acrylate-derived monomers to form a polyacrylate, polyacrylamide, etc.).

The linker can include one or more reaction pairs. In one embodiment, the reaction pair is one of a click-chemistry reaction pair, which can include a first click-chemistry group and a second click-chemistry group that reacts with that first click-chemistry group. Exemplary click-chemistry groups include, e.g., a click-chemistry group, e.g., one of a click-chemistry reaction pair selected from the group consisting of a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; and a splint ligation reaction with a phosphorothioate group and an iodo group; and a reductive amination reaction with an aldehyde group and an amino group.

The linker can include one or more reactive groups. Exemplary reactive groups include an amino (e.g., —NH₂), a thio (e.g., a thioalkoxy group or a thiol group), a hydroxyl, an ester (e.g., an acrylate), a carboxyl (e.g., —CO₂H or a deprotonated form thereof), an imido (e.g., a maleimido or a succinimido), an epoxide, an isocyanate, an isothiocyanate, an anhydride, an amido, a carbamido (e.g., a urea derivative), an azide, an optionally substituted alkynyl, or an optionally substituted alkenyl.

In other embodiments, the linker can include a binding reaction signature, which arises from reacting a binding reaction pair. Exemplary binding groups and binding reaction pairs include those for forming a covalent bond between biotin and avidin, biotin and streptavidin, biotin and neutravidin, desthiobiotin and avidin (or a derivative thereof, such as streptavidin or neutravidin), hapten and an antibody, an antigen and an antibody, a primary antibody and a secondary antibody, and lectin and a glycoprotein.

### EXAMPLES

### Example 1: Overcoming ALK Resistance with Covalent Cysteine-Reactive Inhibitors

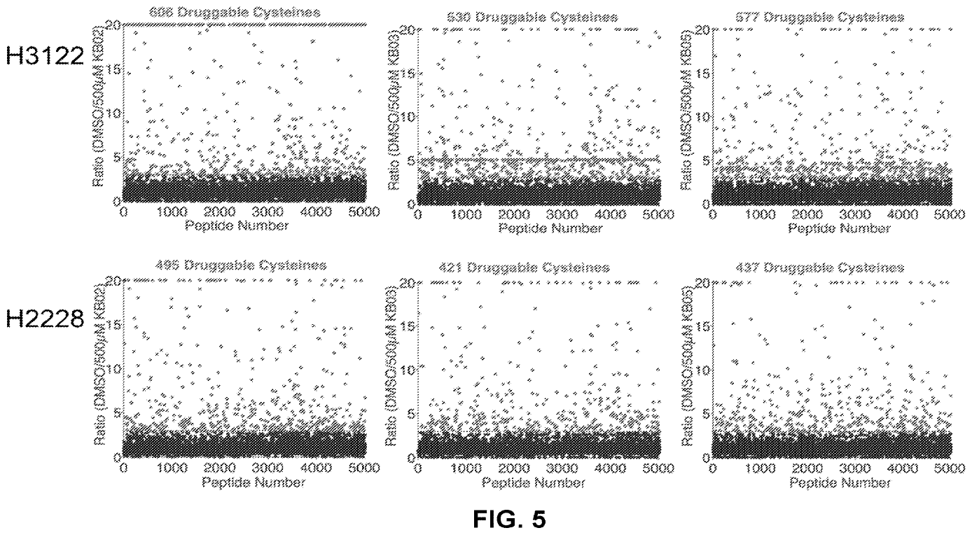

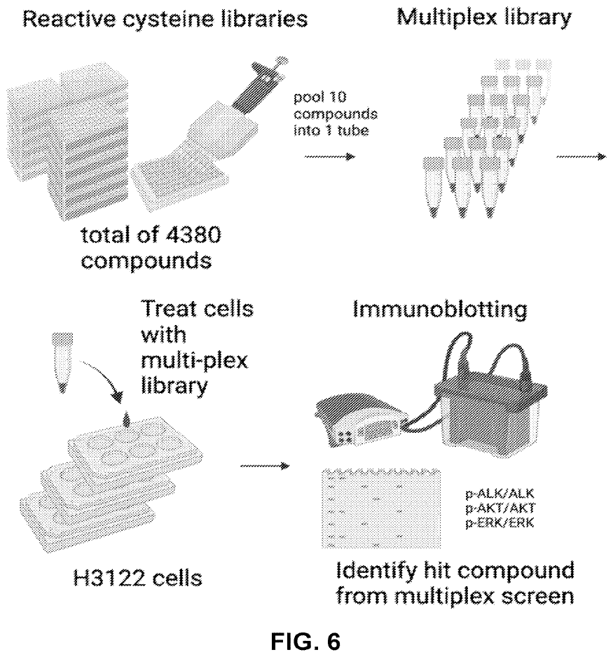

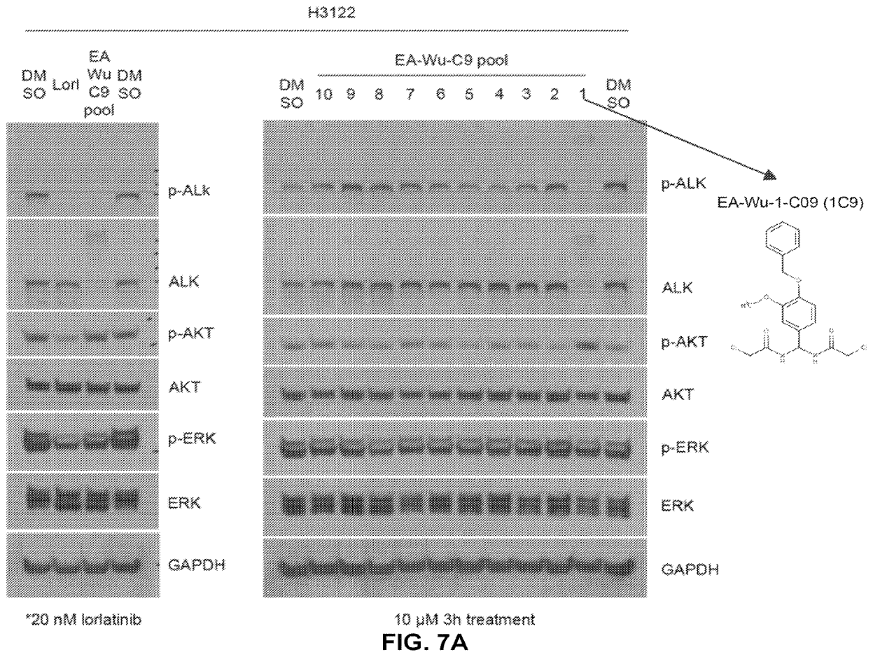

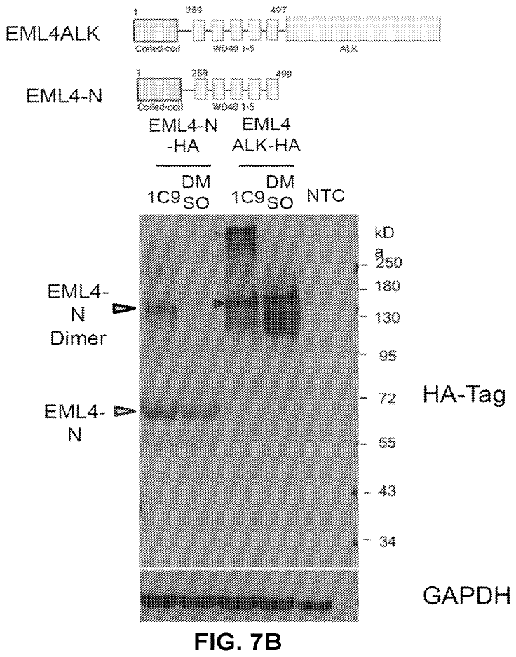

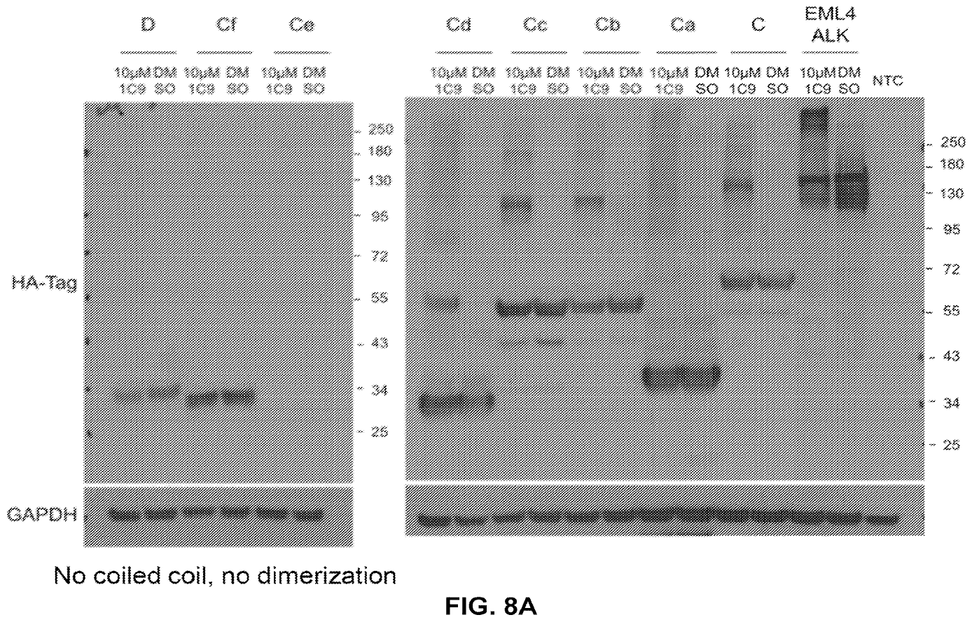

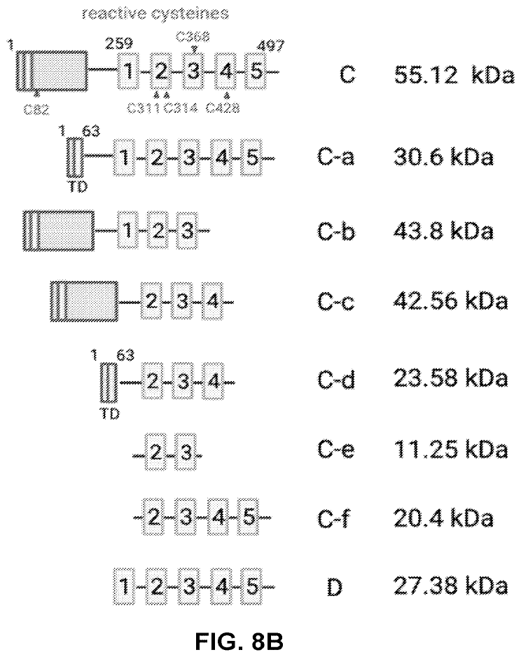

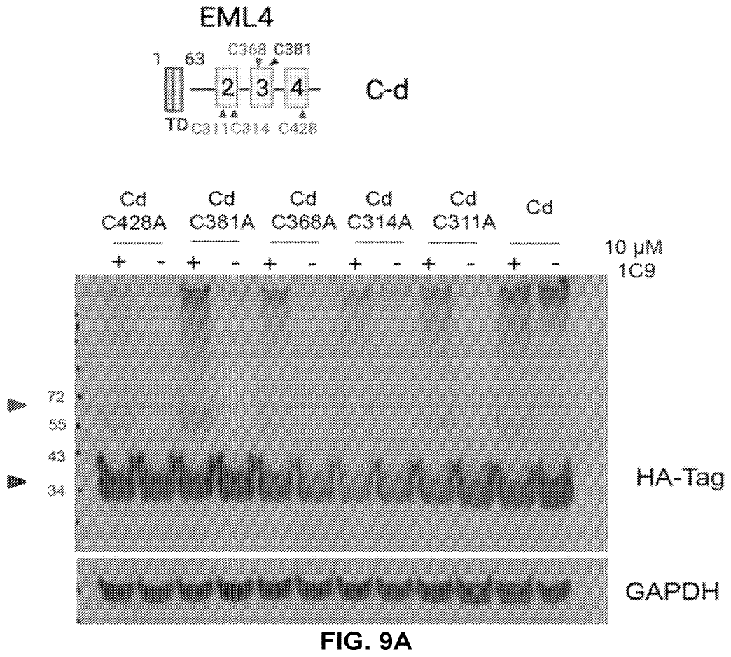

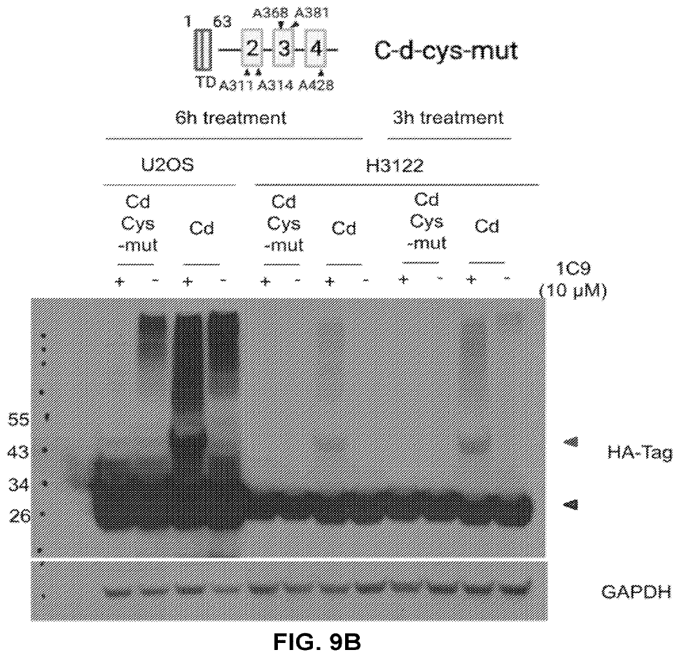

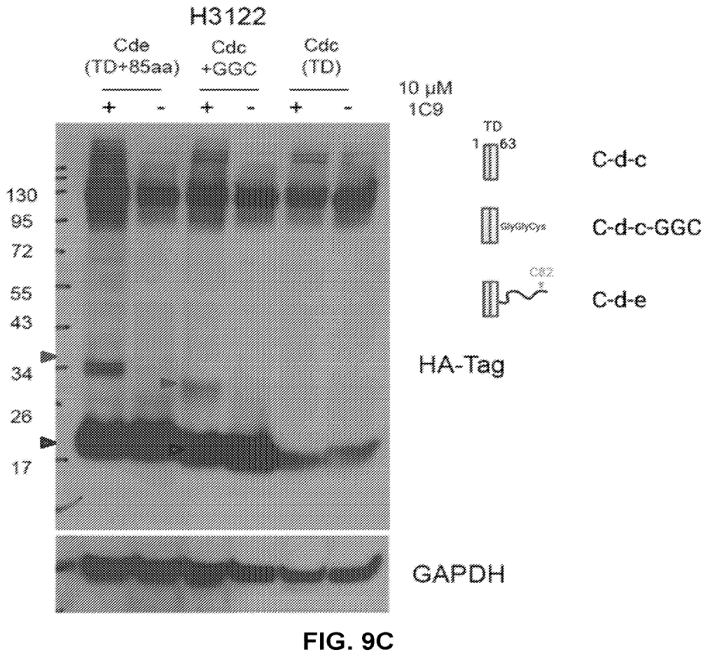

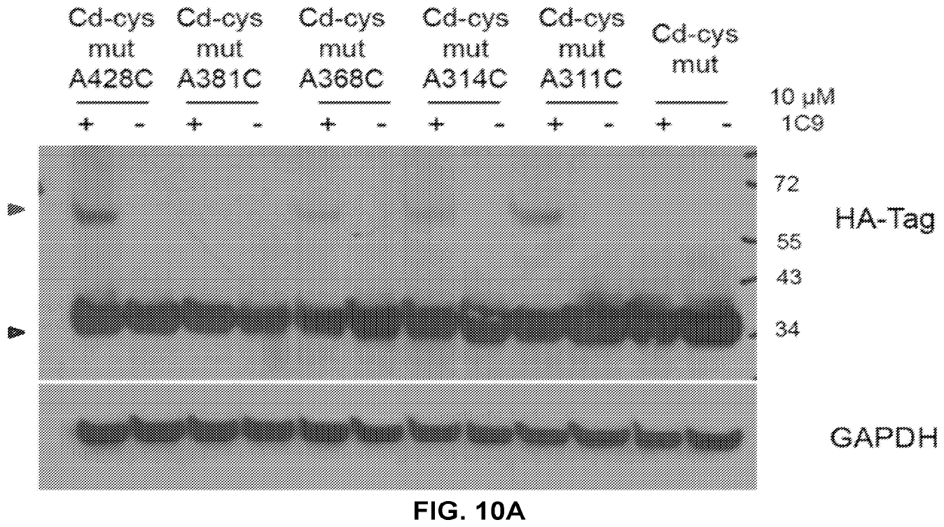

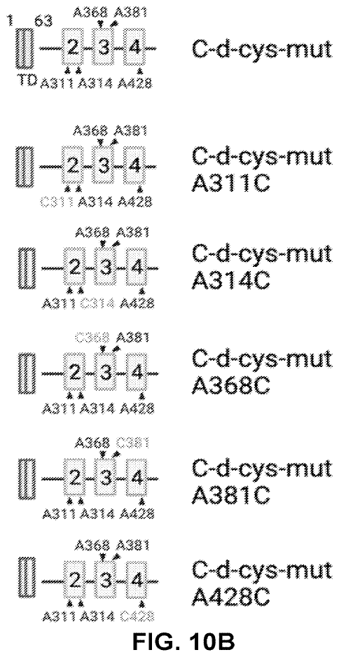

Cysteine druggability mapping (CDM) was used to profile ALK fusion cell lines and patient tissues (FIG. 5). A small molecule screen was performed with a library (e.g., +2,500) of advanced cysteine reactive inhibitors to identify compounds that bind covalently to targets identified in CDM analysis (FIG. 6). In particular, compound 1C9 was identified, and its mechanism of action in dimerizing EML4 was investigated (FIG. 7A-7B). The interaction of EML4-ALK domains with 1C9 was studied. In particular, the coiled-coiled domain of EML4 is present during dimerization (FIG. 8A-8B). In addition, at least one cysteine in the construct is present during dimerization (FIG. 9A-9C). Reactive cysteines in EML4 were tested with 1C9 by alanine mutation of cysteine positions (FIG. 10A-10B).

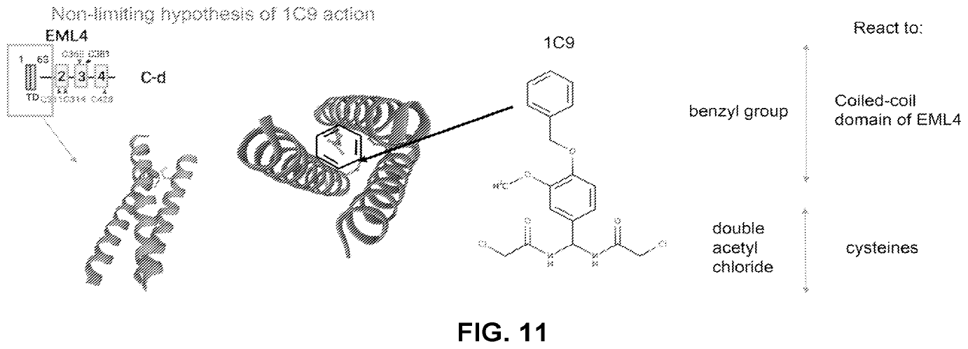

FIG. 11 provides a non-limiting hypothesis of 1C9 action. Without wishing to be limited by mechanism or theory, an aromatic group (e.g., such as benzyl, phenyl, and the like) can act as a specificity group that binds to a specific target site. For EML4, the specific target site can include the coiled-coiled domain. In addition, at least two reactive moieties (e.g., acetyl halides, alkenyl, alkynyl, an electrophilic group, and the like) can form covalent bonds with respective reactive sites for the target. For EML4, the reactive sites can include cysteine. In turn, the reactive moiety can be any that forms a covalent bond with cysteine. In this way, compounds can be designed to have any combination of specificity group(s) and reactive moiety(ies) to binds to and covalently react with a target of interest.

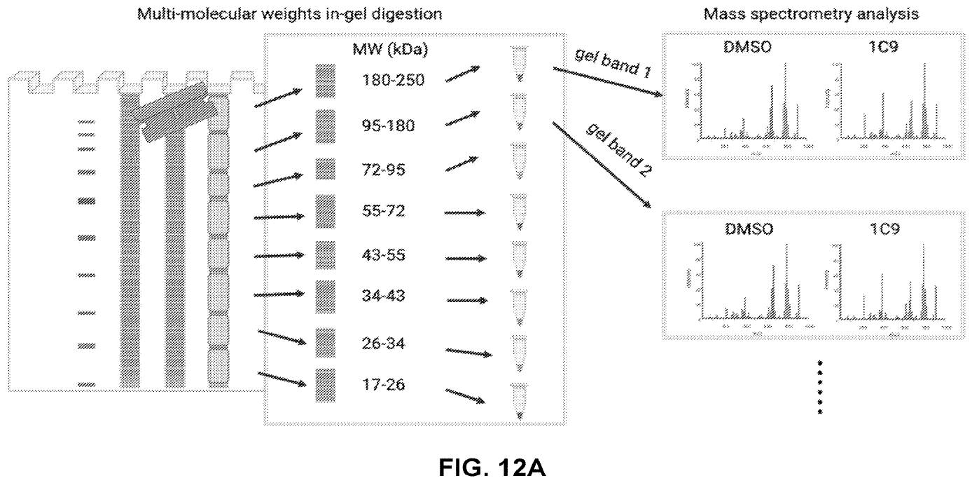

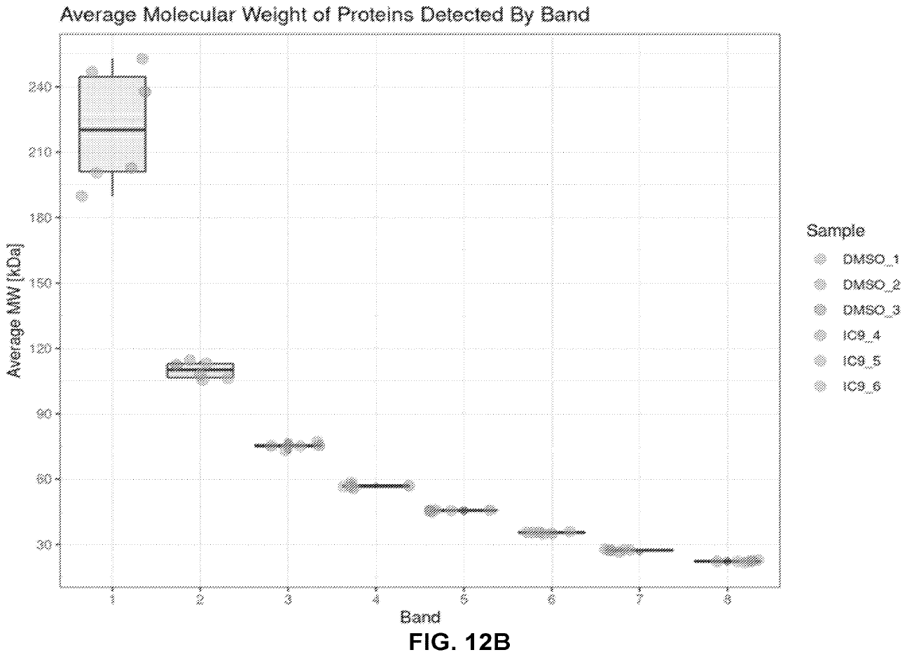

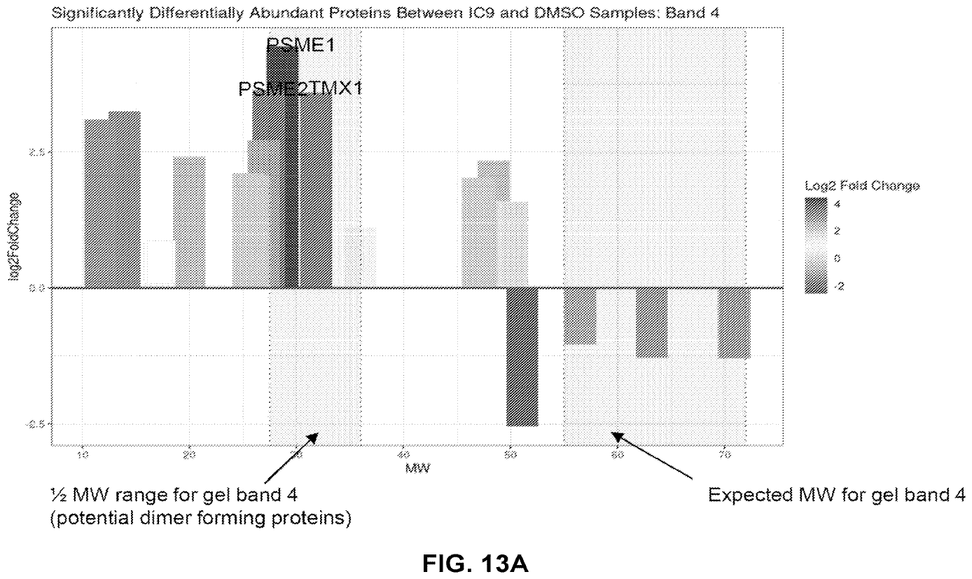

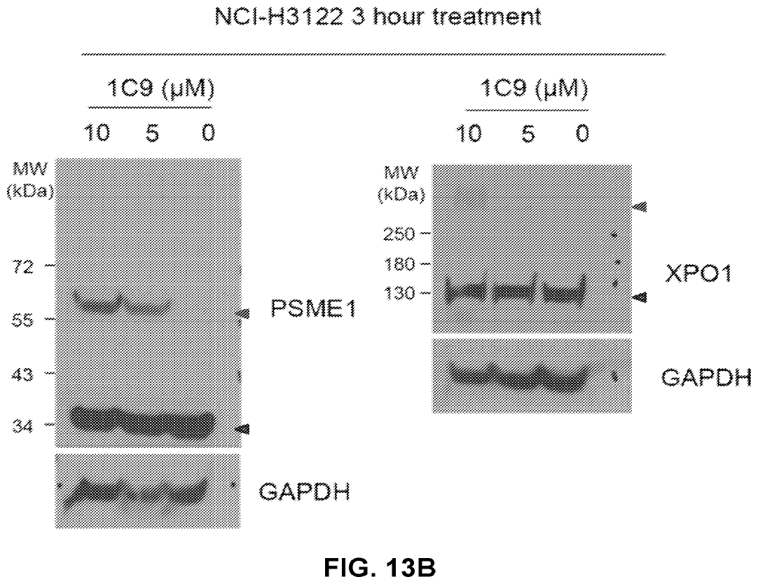

Off-target binding and dimerization of 1C9 was determined by gel-based proteomic analysis (FIG. 12A-12B). Results are provided in FIG. 13A-13B, in which PSME1, PSME2, and TMX1 constitute potential dimer-forming proteins.

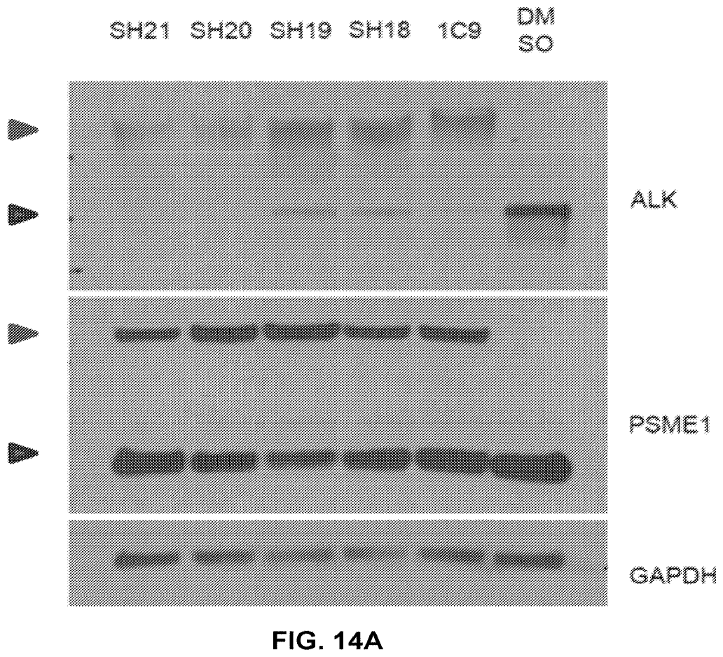

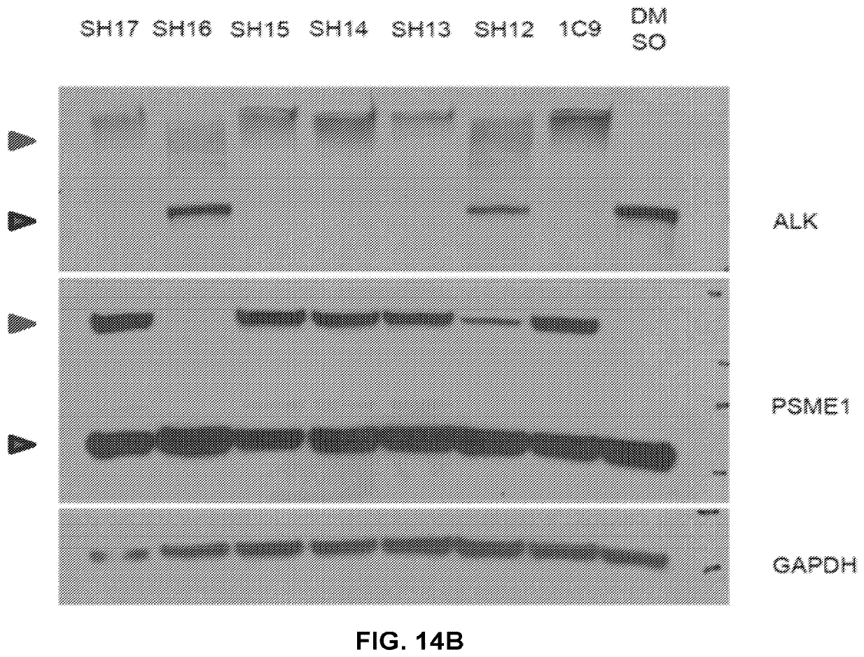

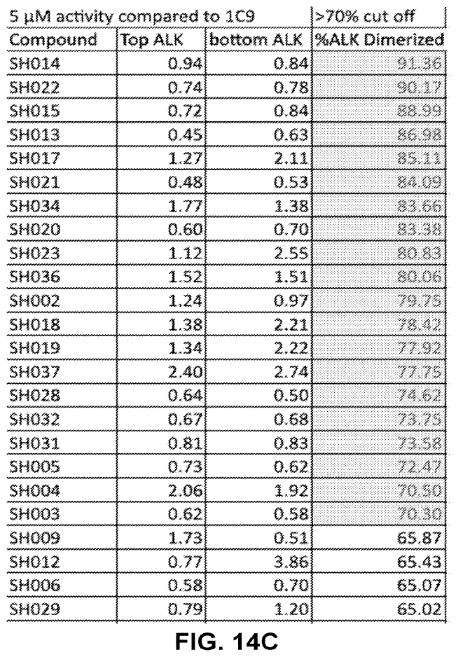

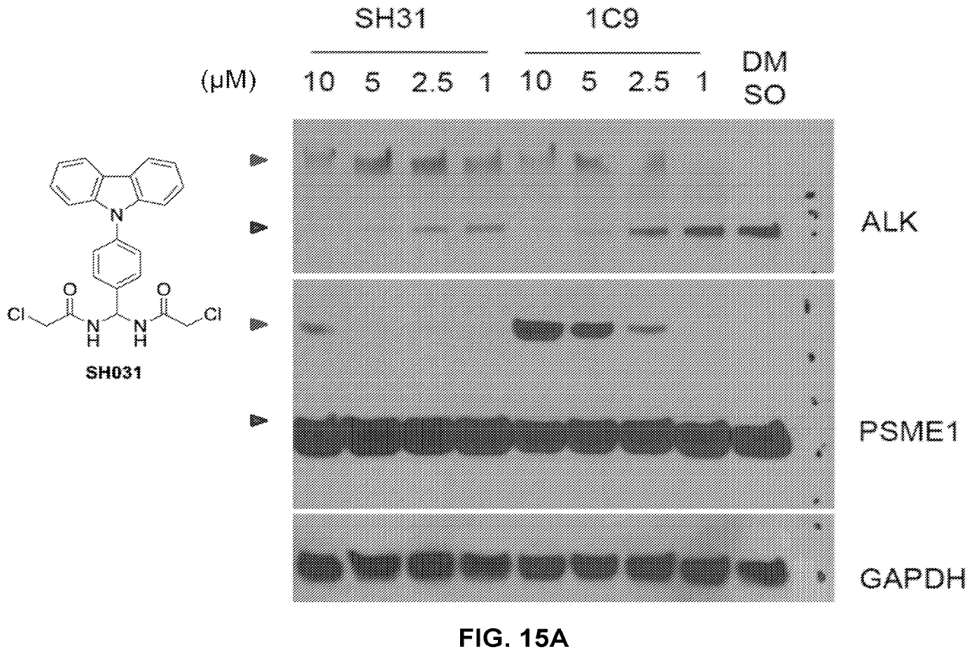

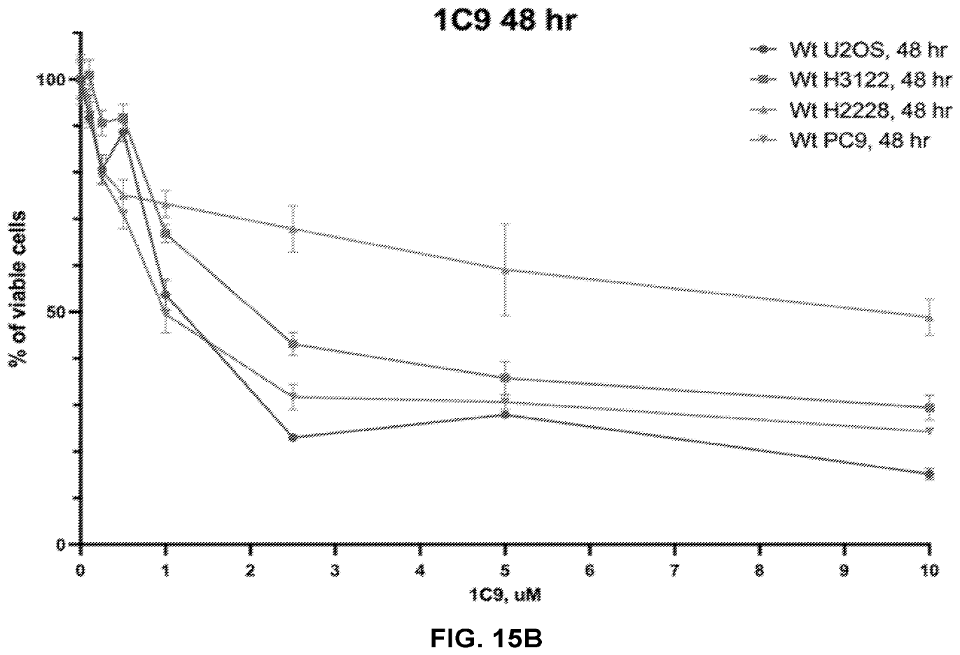

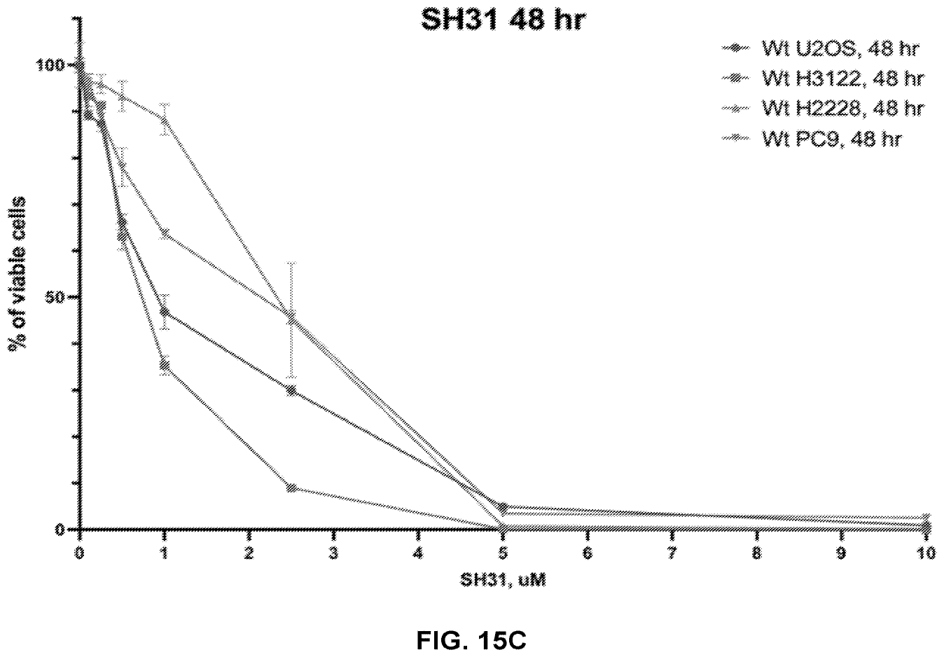

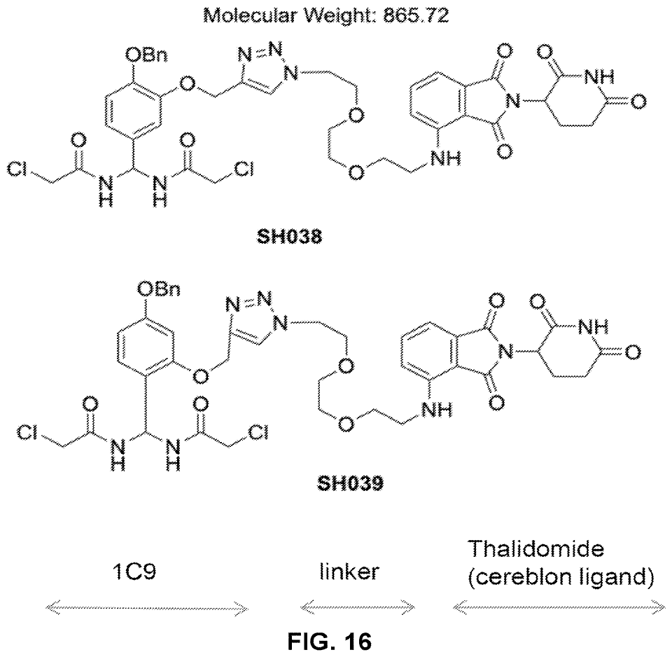

Custom chemical synthesis and screening of a double warhead library were performed (FIG. 14A-14C). ALK cysteine-reactive compounds were validated by assaying cell killing in cell models and in primary patient-derived ALK positive lung cancer lines (FIG. 15A-15C). FIG. 16 shows a non-limiting schematic of a PROTAC including 1C9 with a linker and a cereblon ligand.

Further studies (e.g., to facilitate the hit-to-lead process) can include one or more of the following: determining the structure of 1C9 and EML4 covalently bound complex; guided with the 1C9-EML4 structure, optimizing and/or improving the potency of EML4 binder; exploring the effectiveness of PROTAC in degrading ALK; and/or evaluating the cytotoxicity and cell signaling impact of the optimized compounds.

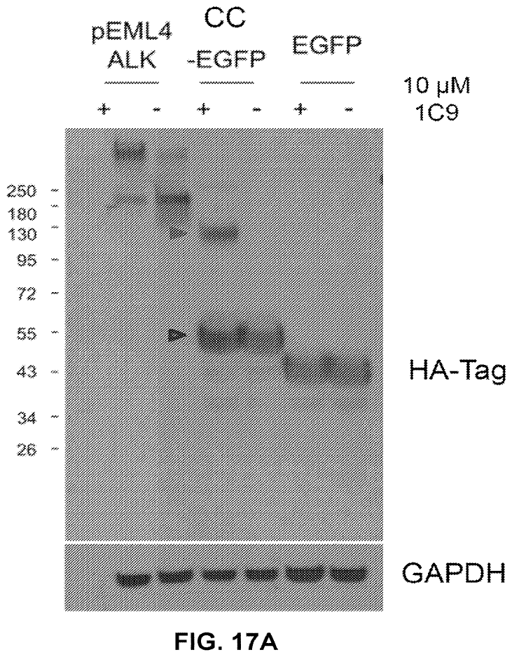

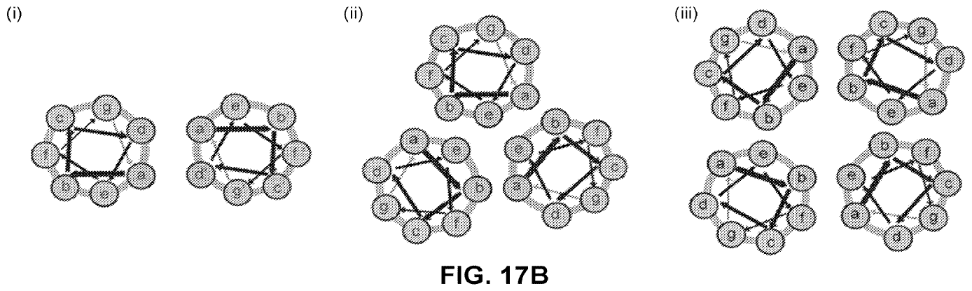

In addition, a covalently-bound dimerizing system can be designed and engineered. 1C9 may dimerize any candidate protein containing the EML4 coiled-coil domain and cysteine. As seen in FIG. 17A, adding the coiled-coil (CC) of EML4 to EGFP exhibited dimerization. In addition to dimers, trimers and tetramers (see, e.g., FIG. 17B) can be designed by mutating and changing the amino acid pairing in the coiled-coil domain. In this way, multimers (e.g., dimers, trimer, tetramers, etc.) of coiled-coil domains can be engineered, and such engineered domains can be attached to any candidate protein. In turn, compounds that target such engineered domains can be designed, identified, tested, and validated. For instance, compounds can include one or more specificity groups that bind to a specific target site in the engineered domain, as well as one or more reactive moieties that react with and form covalent bonds with reactive sites in proximity to the engineered domain.

### Example 2: Overcoming ALK Resistance with Covalent Inhibitors in Lung Cancer

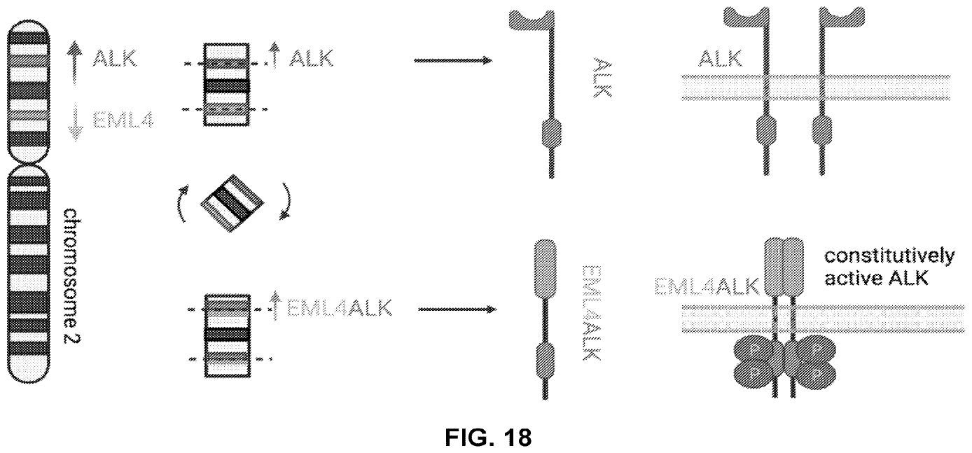

The EML4-ALK fusion gene (see, e.g., FIG. 18) is the main oncogenic driver in 5% of non-small cell lung cancer and has been effectively targeted using ALK-specific tyrosine kinase inhibitors (ALK TKI). While survival rates have improved steadily with each new generation of ALK TKI, resistance generally develops, including second site ALK kinase mutations as well as by-pass track activation. Since almost all anti-ALK approaches have focused on kinase domain inhibition, described herein are inhibitors with alternative mechanisms of action against ALK to overcome current drug resistance.

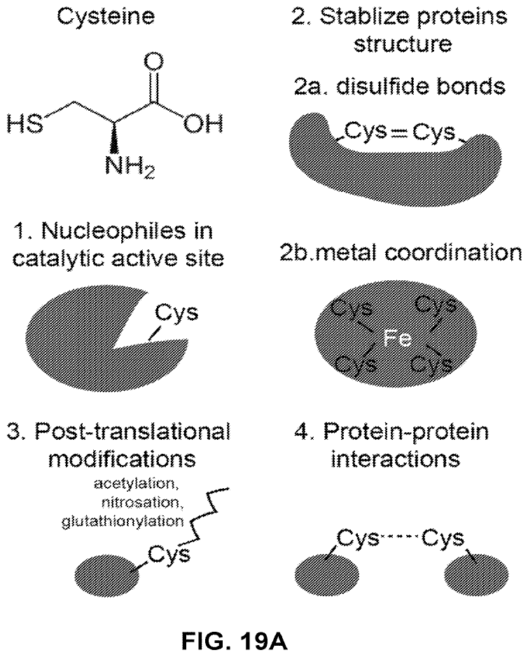

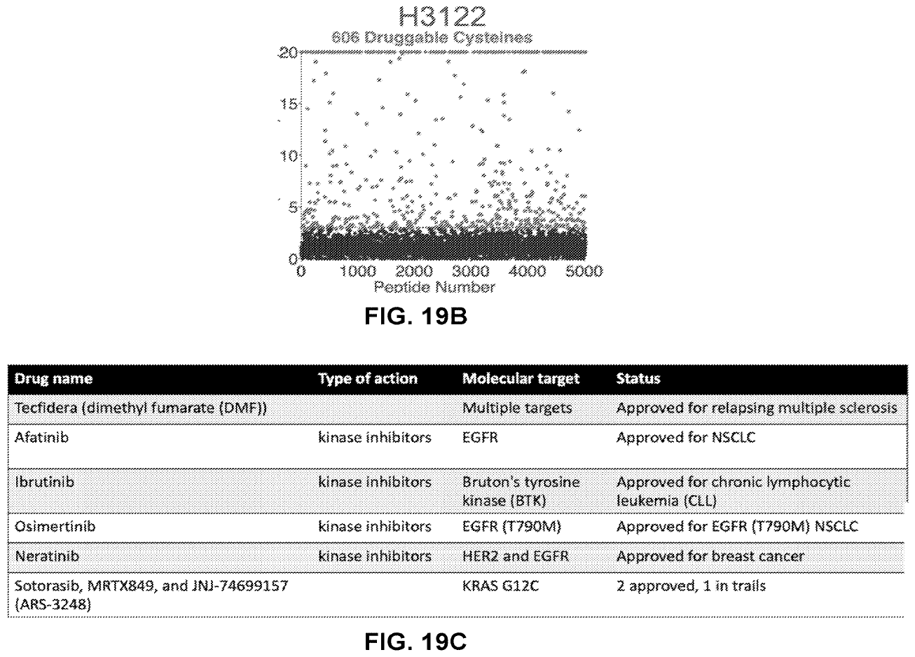

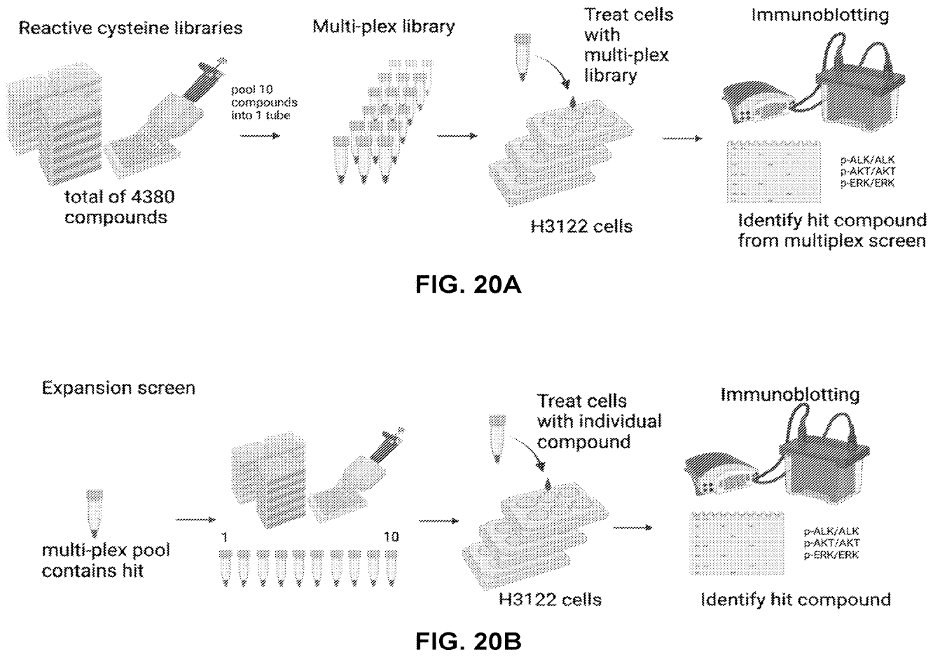

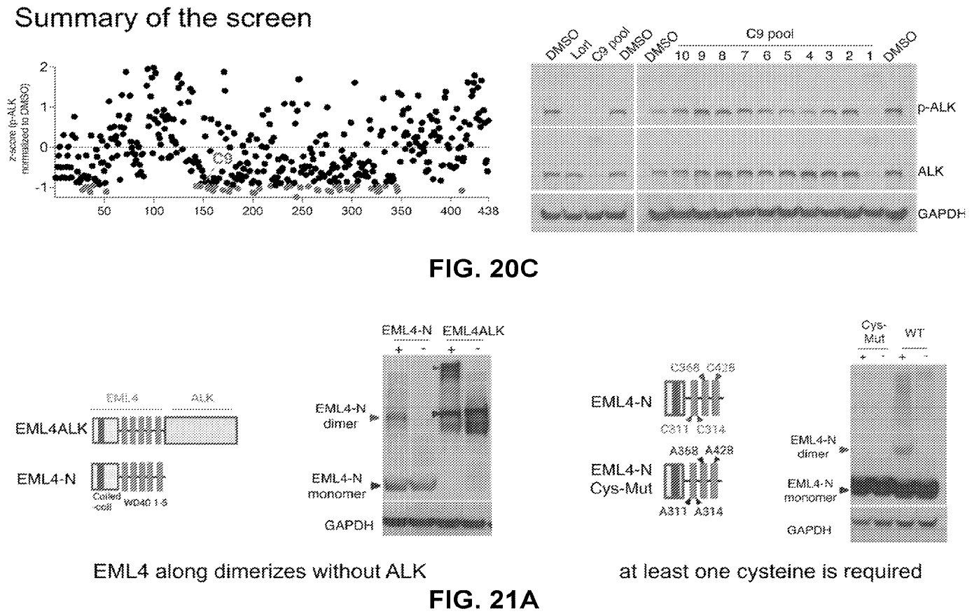

Cysteine-reactive small molecule drugs may provide new mechanisms of target inhibition (FIG. 19A-19B), and clinically successful examples of recent FDA-approved drugs in NSCLC include afatinib, ibrutinib, osimertinib, sotorasib, and adagrasib (see, e.g., FIG. 19C). Described herein are cysteine-reactive covalent inhibitors of EML4-ALK that covalently bind to ALK and inhibits its activation. Using cysteine druggability mapping (CDM), a proteome-wide mass spectrometry target engagement assay, the landscape of reactive cysteines in ALK-positive cell lines was determined, thereby identifying various targetable cysteines within the MAPK pathway and within the EML4-ALK fusion protein itself. To identify small molecules that modulate ALK activation, degradation, and downstream signaling, a high-content phosphorylated protein screen was conducted by testing 4,400 reactive cysteine compounds in a multiplex library including 10 compounds per pool (FIG. 20A) and in an expanded library including individual compounds (FIG. 20B). About 20 potential hits were identified that impacted ALK phosphorylation or expression (FIG. 20C).

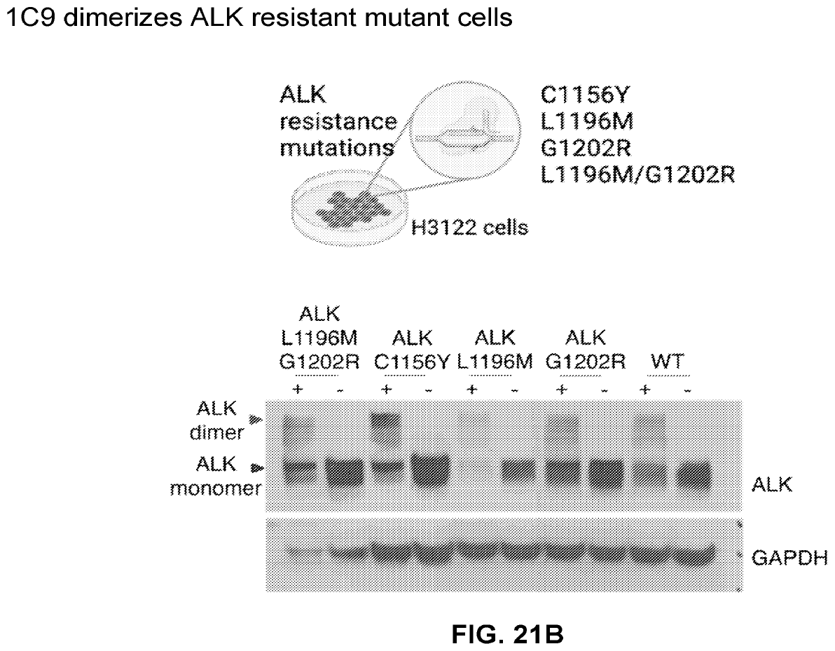

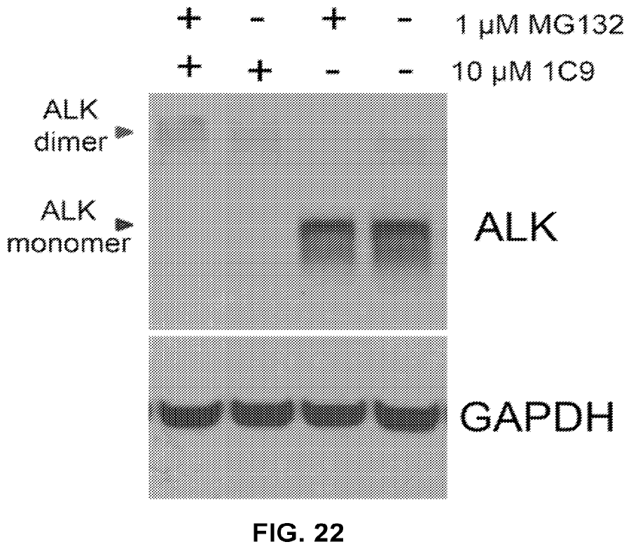

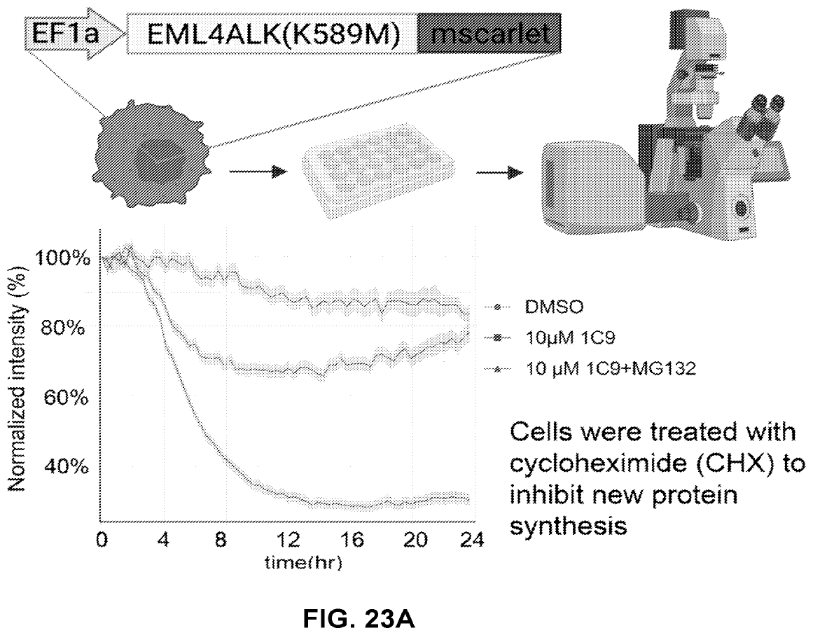

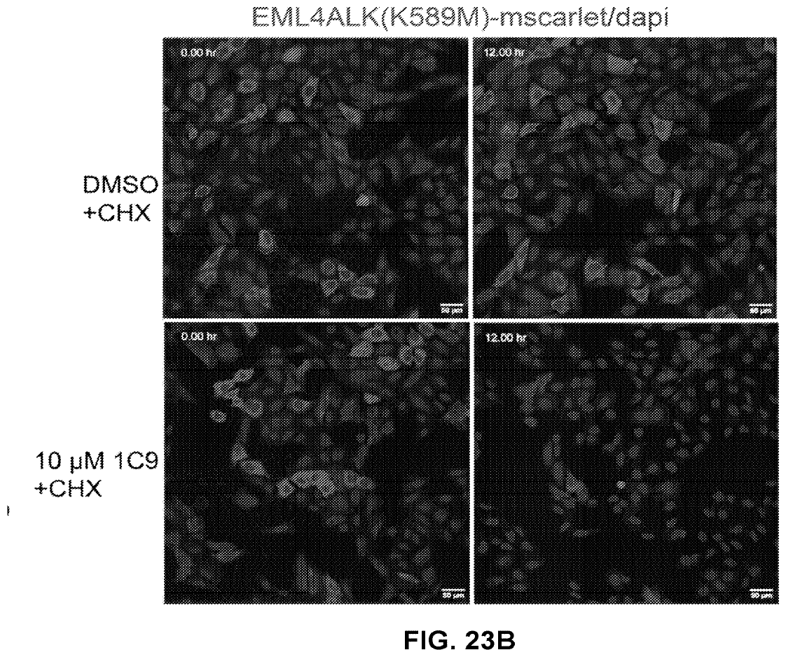

The mechanism of action of 1C9 was further studied, in which EML4 appears to be present during dimerization and at least one cysteine is present during dimerization (FIG. 21A). In addition, use of 1C9 dimerized ALK resistant mutant cells (FIG. 21B). A decrease in ALK expression with 1C9 treatment was observed over time, which led to a non-limiting hypothesis that 1C9 dimerized (or crosslinked) EML4ALK, and this may result in degradation (FIG. 22). Live-cell imaging was conducted at the single-cell level within 24 hours of treatment; and loss of ALK expression over time was observed, while adding the proteasome inhibitor restored the expression (FIG. 23A-23B).

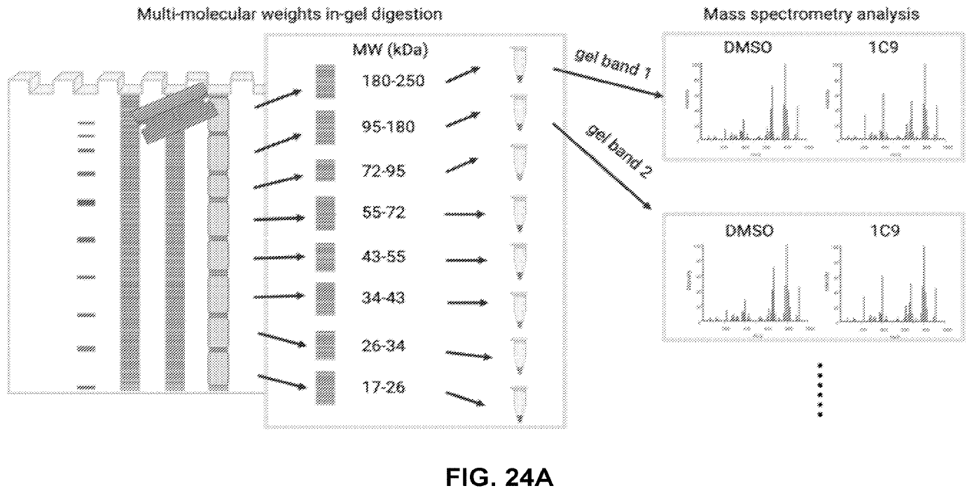

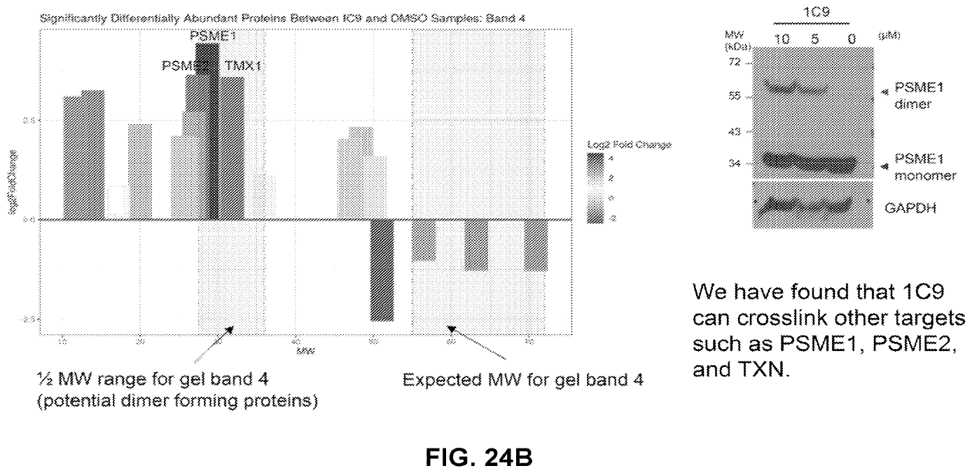

Using proteomic analysis, the comparative specificity of compounds were determined and ranked based on p-ALK reduction efficiency and on their off-target binding patterns. Gel-based proteomic experiments allow for determination of crosslinking by analyzing protein molecular weight shift within the proteome (FIG. 24A-24B).

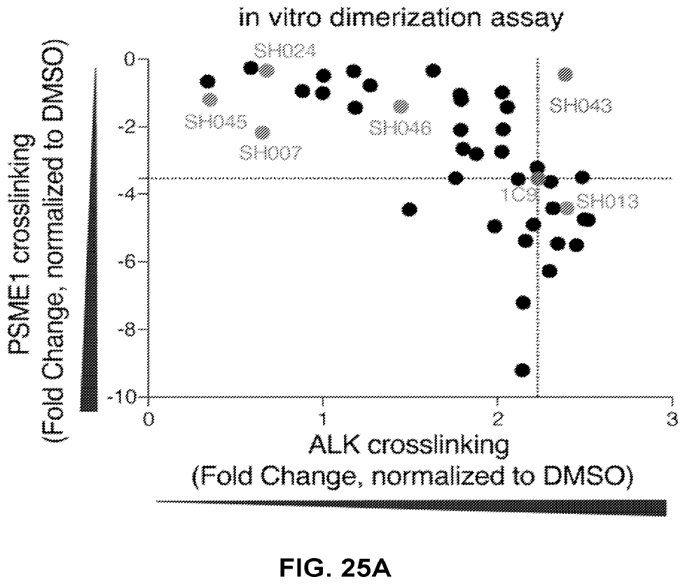

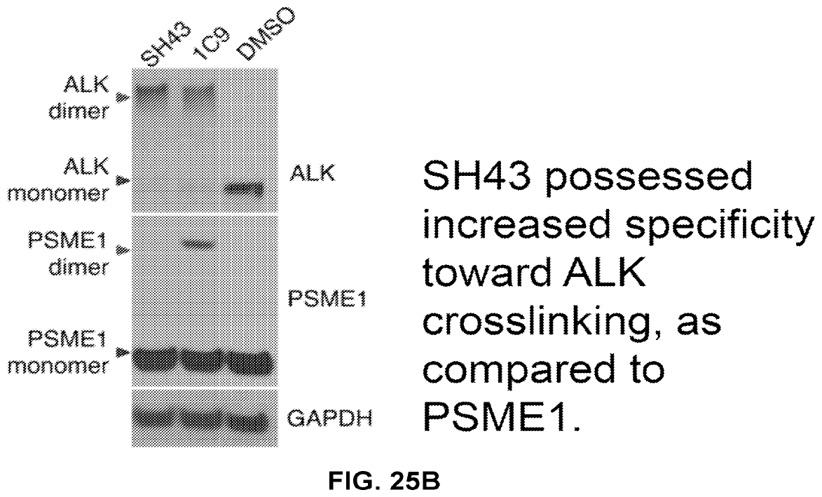

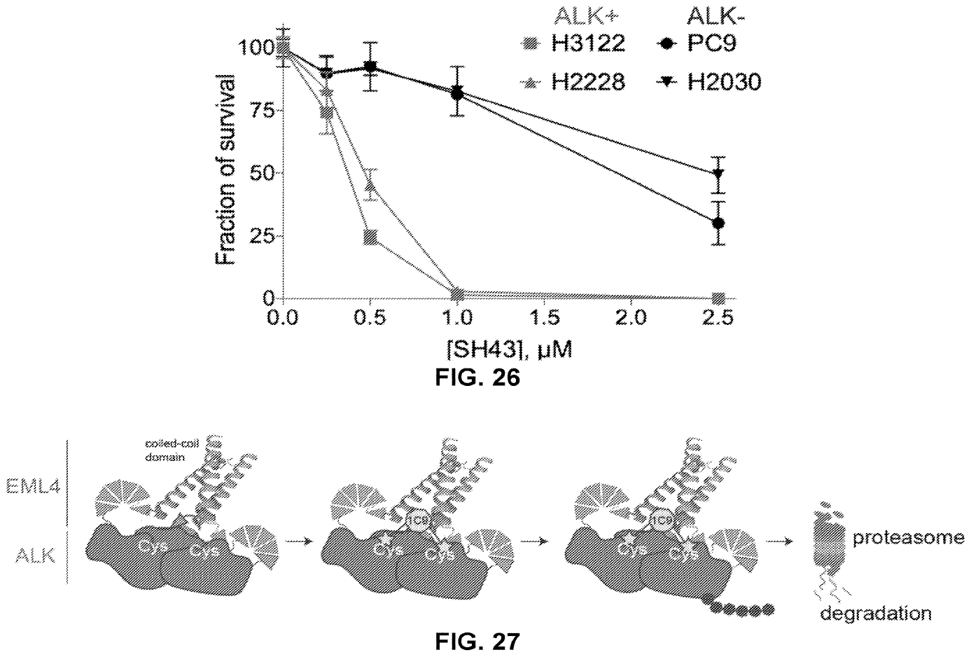

In order to improve target specificity and potency, a chemical library was developed and screened (FIG. 25A). In particular, SH043 possessed increased specificity toward ALK crosslinking, as compared to PSME1 (FIG. 25B). The cell viability assay shows that SH043 presents more toxicity toward ALK-positive lung cancer cells, as compared to ALK-negative cells (FIG. 26). One non-limiting, working model is that a compound can be configured to bind covalently to at least one cysteine in EML4-ALK and can facilitate crosslinking (e.g., dimerization, trimerization, etc.) of the EML4-ALK, resulting in its degradation (FIG. 27).

Hit-to-lead development can include identifying a compound that binds to the EML4 portion of the fusion, and its mechanism of action can be assessed. To further optimize the potency of the compound, a library of compounds (e.g., several hundred analog compounds) can be designed, synthesized, and tested. By applying the hit compounds from the screen in ALK TKI-resistant models, the ability of these compounds to overcome ALK resistance can be evaluated. In some embodiments, a EML4-ALK covalent compound can bind and target the EML4 domain, which can be used, e.g., to potentially overcome ALK resistance.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed above), scientific articles, patent publications, and any other documents cited herein are hereby incorporated by reference for the substance of their disclosure.

1. A method of targeting or engaging one or more targets with a chemical compound, the method comprising:
(i) binding a specificity group of the chemical compound to a specific target site, and
reacting at least two reactive moieties of the chemical compound with respective reactive sites to form one or more covalent bonds,
wherein the one or more targets comprise the specific target site and at least two reactive sites; and
optionally wherein the chemical compound comprises a scaffold disposed between the specificity group and the at least two reactive moieties;
or
(ii) providing the chemical compound to the one or more targets, wherein the chemical compound comprises:
a specificity group configured to interact with a specific target site of the one or more targets, and
at least two reactive moieties, wherein each of the at least two reactive moieties is configured to react with a respective reactive site of the one or more targets; and
incubating the chemical compound with the one or more targets under conditions configured to bind the specificity group to the specific target site and to react the at least two reactive moieties with the respective reactive site to form one or more covalent bonds;
or
(iii) providing an effective amount of the chemical compound, wherein the chemical compound comprises a structure having Formula (I); 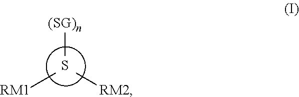
wherein:
S comprises a scaffold:
each SG comprises, independently, a specificity group configured to interact with a respective specific target site of the one or more targets;
each of RM1 and RM2 comprises, independently, a reactive moiety configured to react with a respective reactive site of the one or more targets to form one or more covalent bonds; and
n is an integer of 1 or more).

2.-4. (canceled)

5. A method of treating a disease, the method comprising:
administering a therapeutically effective amount of a chemical compound to a subject in need thereof, wherein the chemical compound comprises a structure having Formula (I): 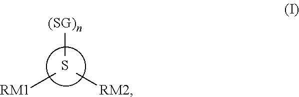
wherein:
S comprises a scaffold;
each SG comprises, independently, a specificity group configured to interact with a respective specific target site of the one or more targets;
each of RM1 and RM2 comprises, independently, a reactive moiety configured to react with a respective reactive site of the one or more targets to form one or more covalent bonds; and
n is an integer of 1 or more.

6.-8. (canceled)

9. The method of claim 1, wherein the specificity group and/or the scaffold, if present, comprises an E3 ubiquitin ligase (E3) ligand.

10. (canceled)

11. The method of claim 1, wherein the chemical compound comprises a structure having Formula (Ia): 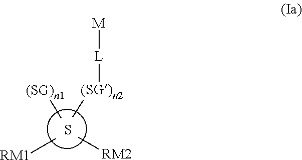
or a pharmaceutically acceptable salt thereof;
wherein:
S comprises a scaffold;
each of SG and SG′ comprises, independently, a specificity group configured to interact with a respective specific target site of the one or more targets;
each L comprises, independently, a linker;
each of RM1 and RM2 comprises, independently, a reactive moiety configured to react with a respective reactive site of the one or more targets to form one or more covalent bonds;
each M comprises, independently, a ligand;
n1 is an integer of 0, 1, or 2;
n2 is an integer of 0, 1, or 2; and
n1+n2 is greater than or equal to 1.

12.-30. (canceled)

31. The method of claim 1, wherein the chemical compound further comprises an E3 ubiquitin ligase (E3) ligand.

32. The method of claim 31, wherein the E3 ligand is a ligand for Skp1-Cullin-F box complex (SCF^{β-TrCP}), von Hippel-Lindau (VHL), murine double minute 2 (MDM2), an inhibitor of apoptosis proteins (IAP), or cereblon (CRBN).

33. The method of claim 31, wherein the E3 ligand comprises a thalidomide-based ligand, a pomalidomide-based ligand, a 4-hydroxythalidomide-based ligand, a lenalidomide-based ligand, a VH032-based ligand, a VHL ligand 1 (VHL A1)-based ligand, a nutlin-3-based ligand, an idasanutlin-based ligand, a bestatin-based ligand, a methyl bestatin-based ligand, or a LCL-161-based ligand.

34. The method of claim 1, wherein the specificity group, SG, and/or SG′, if present, comprises an aromatic group.

35.-37. (canceled)

38. The method of claim 1, wherein the scaffold or S, if present, comprises a monovalent, bivalent, trivalent, tetravalent, or pentavalent linker.

39. (canceled)

40. The method of claim 1, wherein the reactive moiety, the at least two reactive moieties, RM1, and/or RM2, if present, independently comprises an electrophilic group or a leaving group.

41. (canceled)

42. The method of claim 1, wherein the chemical compound comprises a structure having any one of the following formulas: 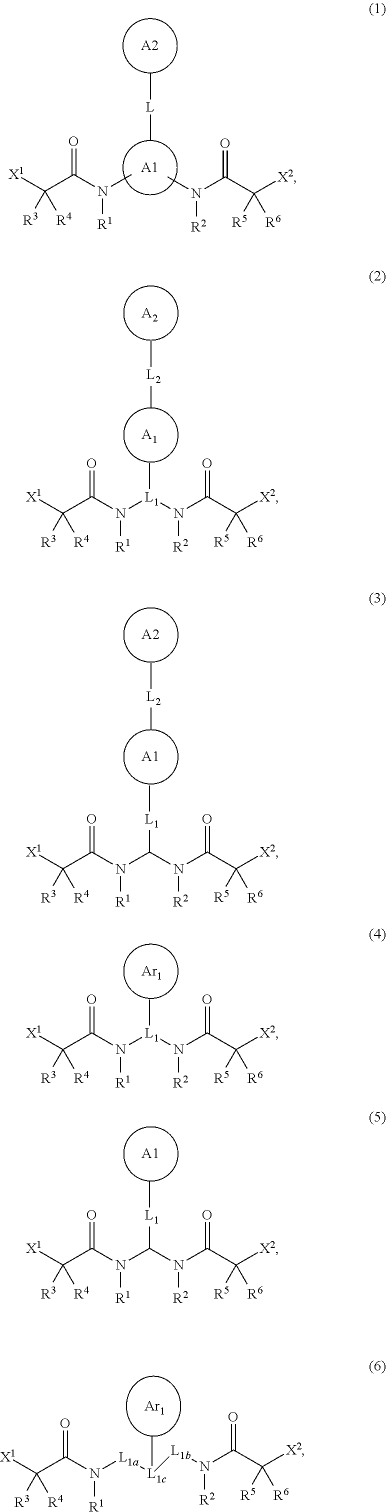 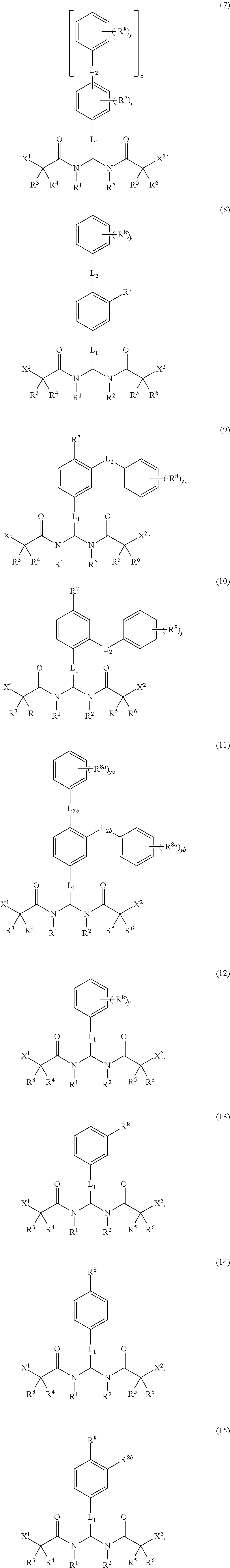 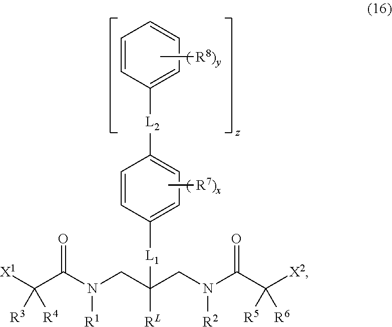
or a pharmaceutically acceptable salt thereof;
wherein:
each of A1, A2, Ar₁, and Ar₂, comprises, independently, one or more aromatic groups;
each of L, L₁, L₁ₐ, L_{1b}, L_{1c}, L₂, L₂ₐ, and L_{2b} comprises, independently, a linker;
each of R¹, R², R³, R⁴, R⁵, R⁶, and R^{L} is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁷, R⁸, R^{8a}, and R^{8b} is, independently, H, optionally substituted C₁₋₁₂ aliphatic, optionally substituted C₁₋₁₂ heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano;
x is an integer of 0-4;
each of y, ya, and yb is, independently, an integer of 0-5; and
z is an integer of 0-4.

43.-47. (canceled)

48. The method of claim 1, wherein the chemical compound comprises a structure having Formula (III): 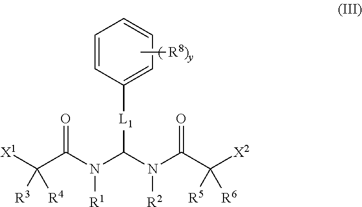
or a pharmaceutically acceptable salt thereof;
wherein:
L₁ comprises a linker;
each of R¹, R², R³, R⁴, R⁵, and R⁶ is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁸ is, independently, H, optionally substituted aliphatic, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
y is an integer of 1-5.

49.-53. (canceled)

54. The method of claim 1, wherein the chemical compound comprises a structure having Formula (IV) or (V): 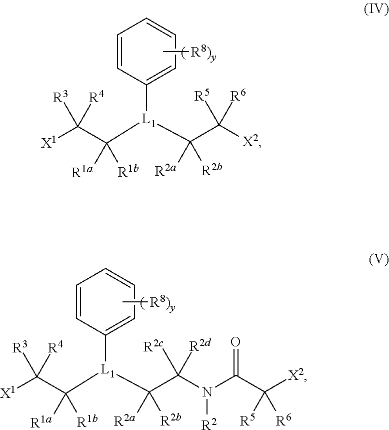
or a pharmaceutically acceptable salt thereof;
wherein:
L₁ comprises a linker;
each of R^{1a}, R^{1b}, R², R^{2a}, R^{2b}, R^{2c}, R^{2d}, R³, R⁴, R⁵, and R⁶ is, independently, H, halo, optionally substituted C₁₋₁₂ aliphatic, or optionally substituted C₁₋₁₂ heteroaliphatic, or wherein Rla and R^{1b}, when taken together, forms an oxo, or wherein R^{2a} and R^{2b}, when taken together, forms an oxo;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁸ is, independently, H, optionally substituted, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
y is an integer of 1-5.

55.-59. (canceled)

60. The method of claim 1, wherein the chemical compound is selected from the group consisting of SH001, SH002, SH003, SH004, SH005, SH006, SH007, SH008, SH009, SH010, SH011, SH012, SH013, SH014, SH015, SH016, SH017, SH018, SH019, SH020, SH021, SH022, SH023, SH024, SH025, SH026, SH027, SH028, SH029, SH030, SH031, SH032, SH033, SH034, SH035, SH036, SH037, SH038, SH039, SH040, SH041, SH042, SH043, SH044, SH045, SH046, SH047, SH048, SH049, SH050, SH051, SH052, SH053, or a pharmaceutically acceptable salt thereof.

61.-63. (canceled)

64. A chemical compound comprising a structure having Formula (II): 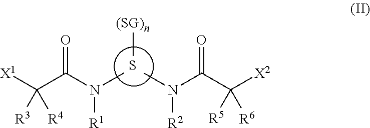
or a pharmaceutically acceptable salt thereof;
wherein:
S comprises a scaffold;
each SG comprises, independently, a specificity group configured to interact with a respective specific target site of one or more targets;
each of R¹, R², R³, R⁴, R⁵, and R⁶ is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
n is an integer of 1 or more, and
wherein;
(i) the compound is not 2-chloro-N-[[(2-chloroacetyl)amino]-(3-methoxy-4-phenylmethoxyphenyl)methyl]acetamide; or
(ii) at least one of S. SG, R¹, R², R³, R⁴, R⁵, or R⁶ comprises an E3 ubiquitin ligase (E3) ligand.

65.-73. (canceled)

74. The chemical compound of claim 64, wherein the chemical compound comprises a structure having Formula (IIa): 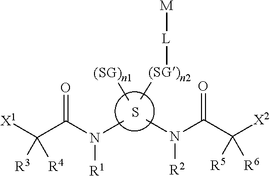
or a pharmaceutically acceptable salt thereof;
wherein:
S comprises a scaffold;
each of SG and SG′ comprises, independently, a specificity group configured to interact with a respective specific target site of the one or more targets;
each L comprises, independently, a linker;
each M comprises, independently, a ligand;
each of R¹, R², R³, R⁴, R⁵, and R⁶ is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group; and
n1 is an integer of 0, 1, or 2;
n2 is an integer of 0, 1, or 2; and
n1+n2 is greater than or equal to 1.

75.-84. (canceled)

85. The chemical compound of claim 64, wherein the chemical compound comprises a structure having any one of the following formulas: 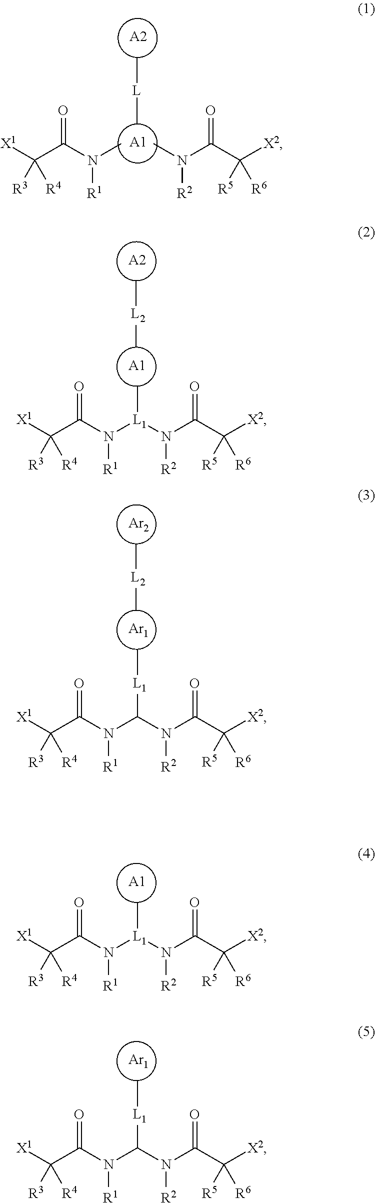 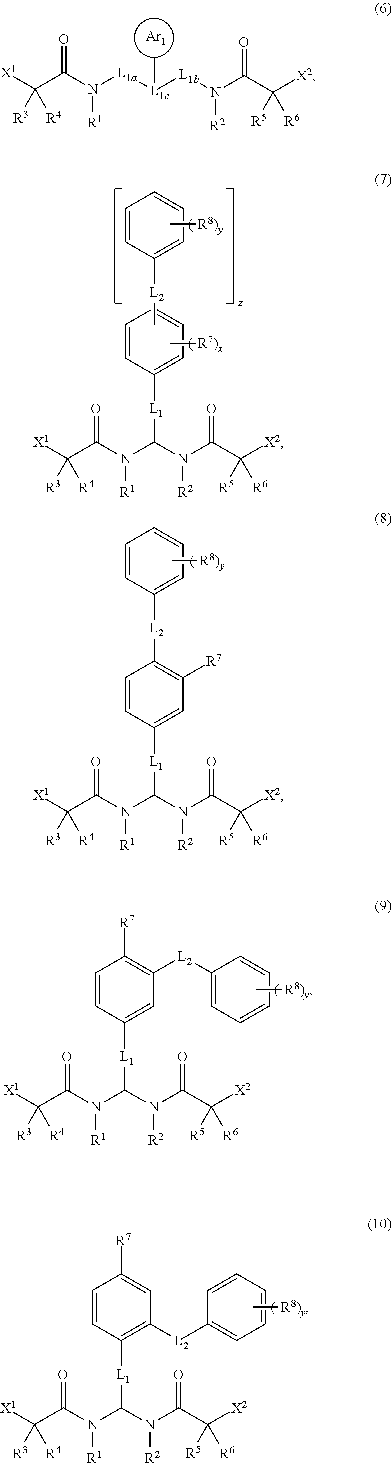 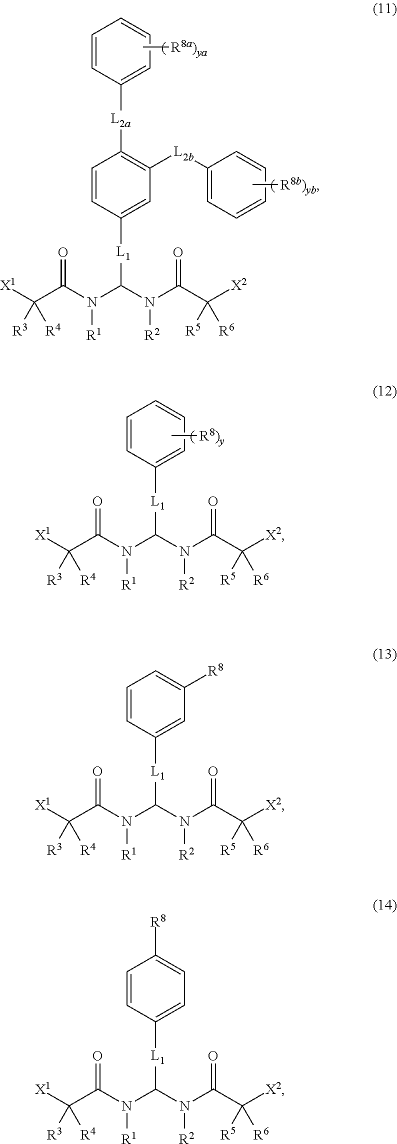 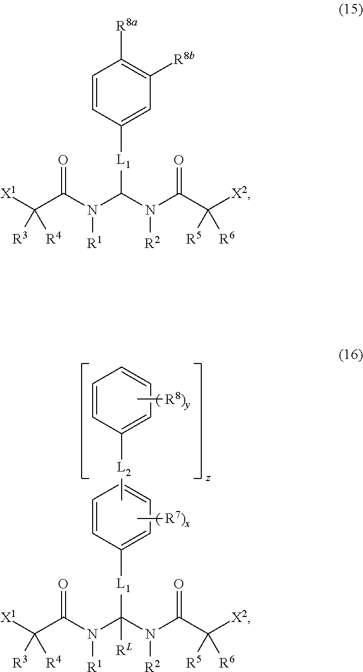
or a pharmaceutically acceptable salt thereof;
wherein:
each of A1, A2, Ar₁, and Ar₂, comprises, independently, one or more aromatic groups;
each of L, L₁, L₁ₐ, L_{1b}, L_{1c}, L₂, L₂ₐ, and L_{2b} comprises, independently, a linker;
each of R¹, R², R³, R⁴, R⁵, R⁶, and R^{L} is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁷, R⁸, R^{8a}, and R^{8b} is, independently, H, optionally substituted C₁₋₁₂ aliphatic, optionally substituted C₁₋₁₂ heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano;
x is an integer of 0-4;
each of y, ya, and yb is, independently, an integer of 0-5; and
z is an integer of 0-4.

86.-90. (canceled)

91. The chemical compound of claim 64, wherein the chemical compound comprises a structure having Formula (III): 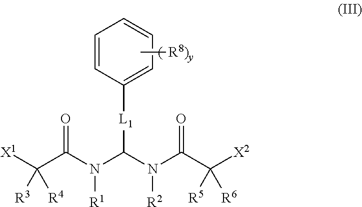
or a pharmaceutically acceptable salt thereof;
wherein:
L₁ comprises a linker;
each of R¹, R², R³, R⁴, R⁵, and R⁶ is, independently, H, halo, optionally substituted C₁₋₁₂ aliphatic, or optionally substituted C₁₋₁₂ heteroaliphatic;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁸ is, independently, H, optionally substituted aliphatic, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
y is an integer of 1-5.

92.-96. (canceled)

97. The chemical compound of claim 64, wherein the chemical compound comprises a structure having Formula (IV) or (V): 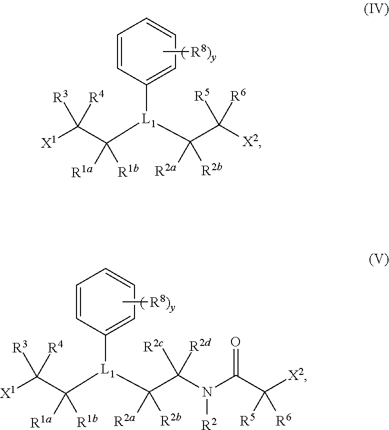
or a pharmaceutically acceptable salt thereof;
wherein:
L₁ comprises a linker;
each of R^{1a}, R^{1b}, R², R^{2a}, R^{2b}, R^{2c}, R^{2d}, R³, R⁴, R⁵, and R⁶ is, independently, H, halo, optionally substituted C₁₋₁₂ aliphatic, or optionally substituted C₁₋₁₂ heteroaliphatic, or wherein R^{1a} and R^{1b}, when taken together, forms an oxo, or wherein R^{2a} and R^{2b}, when taken together, forms an oxo;
each of X¹ and X² is, independently, halo, a leaving group, or an electrophilic group, or wherein X¹ and R³, when taken together with the carbon atom to which each are attached, forms an optionally substituted alkenyl group, or wherein X¹ and R³ and R⁴, when taken together with the carbon atom to which each are attached, forms an optionally substituted alkynyl group, or wherein X² and R⁵, when taken together with the carbon atom to which each are attached, forms an optionally substituted alkenyl group, or wherein X² and R⁵ and R⁶, when taken together with the carbon atom to which each are attached, forms an optionally substituted alkynyl group;
each of R⁸ is, independently, H, optionally substituted, optionally substituted heteroaliphatic, optionally substituted aromatic, optionally substituted aralkyl, optionally substituted alkaryl, optionally substituted heteroaromatic, optionally substituted aryloxy, optionally substituted aralkoxy, optionally substituted aralkanoyl, optionally substituted aralkanoyloxy, optionally substituted aryloyloxy, optionally substituted aryloxycarbonyl, optionally substituted aryloyl, optionally substituted heterocyclyl, halo, hydroxyl, amino, nitro, or cyano; and
y is an integer of 1-5.

98.-156. (canceled)
